(19) United States
(12) Patent Application Publication
(10) Pub. No.: US 2024/0075074 A1
(43) Pub. Date: Mar. 7, 2024

(54) USE OF UMBILICAL CORD BLOOD DERIVED EXOSOMES FOR TISSUE REPAIR

(71) Applicants: Stemlab, SA, Cantannhede (PT); Biocant- Associacao de Transferencia de Technologia, Cantanhede (PT)

(72) Inventors: Lino da Silva Ferreira, Coimbra (PT); Joana CORREIA, Cernache (PT); Renato Manuel Soares CARDOSO, Luso (PT); Maria Helena Henriques Antunes, Coimbra (PT)

(21) Appl. No.: 18/327,463

(22) Filed: Jun. 1, 2023

Related U.S. Application Data
(62) Division of application No. 16/087,559, filed on Sep. 21, 2018, now abandoned.
(60) Provisional application No. 62/313,002, filed on Mar. 24, 2016.
(60) Provisional application No. 62/336,907, filed on May 16, 2016.

(51) Int. Cl. A61K 35/51; A61K 31/7105; A61K 35/15; A61K 35/17; A61P 17/02

(52) U.S. Cl. CPC A61K 35/51; A61K 31/7105; A61K 35/15; A61K 35/17; A61P 17/02

(57) ABSTRACT

The subject invention provides a method for promoting wound healing in a patient in need thereof comprising contacting a wound of the patient with exosomes secreted by umbilical cord blood mononuclear cells (UCBMNCs) so as to thereby promote wound healing in the patient. The subject application also provides a method for promoting wound healing in a patient in need thereof comprising contacting a wound of the patient with a composition comprising one or more miRNA and a pharmaceutically acceptable carrier so as to thereby promote wound healing in the patient.

This application claims priority of U.S. Provisional Application No. 62/336,907, filed May 16, 2016 and U.S. Provisional Application No. 62/313,002, filed Mar. 24, 2016, the entire content of each of which is hereby incorporated by reference herein.

Throughout this application, various publications are referenced, including referenced in parenthesis. Full citations for publications referenced in parenthesis may be found listed at the end of the specification immediately preceding the claims. The disclosures of all referenced publications in their entireties are hereby incorporated by reference into this application in order to more fully describe the state of the art to which this invention pertains.

### BACKGROUND OF INVENTION

Chronic wounds are defined as wounds that do not heal in 3 months (Nuhan et al., 2014). A wound is a disruption of normal structure and function of the skin. In contrast to acute wounds, chronic wounds fail to proceed through an orderly and timely reparative process that would result in sustained restoration of the skin.

In developed countries, it has been estimated that 1-2% of the population will experience a chronic wound during their lifetime (Gottrup, 2004). In addition, there are about 150 million diabetes patients worldwide, of which about 15% suffer from foot ulcerations that can evolve into non-healing chronic wounds (Boulton et al., 2005).

Chronic and non-healing wounds are particularly costly because of the need of several treatments in comparison to acute wound treatments. The burden caused by chronic wounds is growing due to an increase of health care costs, aging of the population and a sharp increase of diabetes and obesity worldwide (Sen, 2009).

The most common types of chronic wounds are: (i) arterial ulcers, (ii) venous ulcers, (iii) diabetic ulcers and (iv) pressure ulcers.

Arterial ulcers are formed in patients with hypertension, atherosclerosis and thrombosis, where the reduced blood supply leads to an ischemic state.

Venous ulcers account for more than half of ulcer cases, and they occur mainly in the lower limbs (mainly the legs) and they are associated with deep vein thrombosis, varicose veins and venous hypertension (Eberhardt & Raffetto, 2005). It is estimated that in the United States, there are approximately 0.5 million patients with venous ulcers (Abbade & Lastoria, 2005).

Diabetic ulcers occur in individuals with diabetes mellitus, who have impaired immune function, ischemia (due to poor blood circulation), and neuropathy (nerve damage). Diabetic neuropathy increases the chance of lower-extremity amputations unless treated. In 2004, about 71,000 non-traumatic lower-limb amputations were performed in people with diabetes (Sen, 2009) and the amputations rates increase with age.

Pressure ulcers occur due to a constant pressure and friction from body weight over a localized area which may lead to breakage of skin and ulceration. Vulnerable patients include aged people, stroke victims, patients with diabetes or dementia, patients with spinal cord injury, patients in wheelchairs or suffering from impaired mobility or sensation. Annually, 2.5 million pressure ulcers are treated in the United States in acute care facilities alone (Reddy et al., 2006).

The Wound Healing Process

Wound healing is a dynamic process that consists of four continuous and overlapping phases: hemostasis, inflammation, proliferation, and tissue remodeling. (Guo et al., 2010). For optimal wound healing, events during each phase must occur in a precise and regulated manner. Interruptions, aberrancies, or prolongation in the process can lead to delayed wound healing or non-healing chronic wounds. (Guo et al., 2010).

The hemostasis phase of wound healing begins immediately after injury and is characterized by vascular constriction, platelet aggregation, degranulation, and fibrin clot formation. (Guo et al, 2010). Specifically, contact with exposed extracellular matrix at the site of injury causes platelets to release clotting factors, leading to the formation of a blood clot. In addition, the contact will also cause platelets to release pro-inflammatory cytokines and growth factors such as transforming growth factor (TGF)-β, platelet derived growth factor (PDGF), fibroblast growth factor (FGF), epidermal growth factor (EGF), and insulin-like growth factor (IGF). These molecules activate and attract neutrophils and macrophages thereby initiating the inflammatory phase of wound healing. These molecules also attract endothelial cells and fibroblasts, which will affect the later phases of proliferation and tissue remodeling. (Guo et al., 2010; Velnar et al., 2009).

The inflammatory phase of wound healing is characterized by neutrophil infiltration, monocyte infiltration and differentiation into macrophage, and lymphocyte infiltration. Neutrophils primarily function is to clear, via phagocytosis, invading microbes and cellular debris in the wound site by producing substances such as proteases and reactive oxygen species (ROS). Macrophages carry out several functions in the wound healing process, including releasing cytokines to recruit and activate leukocytes and clearing apoptotic cells (including neutrophils). Macrophages also release growth factors such as TGF-β and FGF, which activates keratinocytes, fibroblasts, and endothelial cells thereby initiating the proliferative phase of wound healing. Lymphocytes also migrate into wounds during late inflammatory phase, however, their role wound healing is not completely understood. (Guo et al., 2010; Velnar et al., 2009).

The proliferative phase is characterized by re-epithelialization, angiogenesis, collagen synthesis, and extracellular matrix formation. In the reparative dermis, fibroblasts and endothelial cells are the most prominent cell types present and they function to support capillary growth, collagen formation, and the formation of granulation tissue at the site of injury. (Guo et al, 2010; Velnar et al., 2009).

The remodeling phase is the final phase of wound healing and it may last up to one or two years, sometimes for an even more prolonged period of time. The remodeling phase is a delicate balance between degradation and synthesis and is characterized by collagen remodeling and vascular maturation and regression. Specifically, the extracellular matrix is remodeled into an architecture which approaches that of the normal tissue, and the wound undergoes contraction mediated by myofibroblasts. (Guo et al., 2010; Velnar et al., 2009).

Diabetes Mellitus

Diabetes mellitus represents a spectrum of metabolic disorders characterized by chronic hyperglycemia. Type I diabetes mellitus, also known as juvenile-onset diabetes, results from the autoimmune destruction of insulin-secreting pancreatic β-cells. Type II diabetes mellitus, also known as adult-onset diabetes, is characterized by excessive insulin secretion, tissue insulin resistance, and subsequent β-cell dysfunction. (Goutos et al., 2015).

Effect of Diabetes Mellitus on the Wound Healing Process

Diabetic individuals exhibit impaired healing of acute wounds, and are prone to develop chronic wounds, such as diabetic foot ulcers. (Guo et al., 2010).

Diabetes mellitus exerts a detrimental effect on several aspects of the wound healing process. For example, diabetes alters the balance between different phenotypes of macrophages which causes increased number of inflammatory cells and hinders the transition into the proliferative phase. Diabetes is also associated with reduced levels of growth factors at the wound site, abnormal extracellular matrix deposition, and inhibition of vascularization. (Goutos et al., 2015; Guo et al., 2010).

Exosomes

Exosomes are liposome-like vesicles (30-200 nm) secreted by most types of cells, which are formed inside the secreting cell in compartments referred as multivesicular bodies (MVBs), and are subsequently released by fusion of the endosomal compartment with the plasma membrane, resulting in content release to the extracellular milieu (Raposo and Stoorvogel, 2013). They contain specific sets of proteins, lipids and RNA, depending on the type of secreting cell and stimuli, and not a random sample of cytoplasmic content (Raposo and Stoorvogel, 2013; Mathivanan S. and Simpson R. J., 2009). They carry genetic material, in the form of mRNA and microRNA, a feature that promoted them to promising biologically-derived gene-delivery systems (O'Loughlin et al., 2012).

Cell types employed in experimental regeneration of injured tissues, such as mesenchymal stem cells (MSC) or hematopoietic stem cells (HSC), are rich sources of exosomes, which have been proposed to successfully replace cell-based therapies in different injury models (Bian et al., 2014; Shabbir et al., 2015; Zhang B. et al., 2015; Zhang J. et al.; Lai R C, 2010; Zhou Y, 2013; Sahoo S, 2011).

MSC are multipotent, non-hematopoietic adult stem cells, which can be isolated from umbilical cord (Erices et al., 2000; Gang et al., 2004), placental or adipose tissue, and bone marrow. These cells can differentiate into several cell types such as osteoblasts, chondrocytes, adipocytes and endothelial cells (Pittenger et al., 1999; Crapnell et al., 2013). It has been recently demonstrated that, in addition to their direct regenerative potential through differentiation, these cells can induce repair by paracrine stimulation, such as exosomes secretion (Lai et al., 2011; Lai et al., 2010). Exosomes secreted by MSC have been shown to induce repair in different mice models of disease, such as myocardial infarction (Lai et al., 2010) and also wound healing (Zhang et al., Stem Cells 2014; Shabir et al., Stem Cells Dev 2015; Zhang et al. J Transl Med 2015).

Despite many papers demonstrating that exosomes derived from MSC can induce tissue regeneration such as wound healing, the same does not hold true for exosomes derived from Umbilical Cord Blood Mononuclear Cells (UCBMNCs). Furthermore, the bioactivity of exosomes secreted by human Umbilical Cord Blood mononuclear cells has not been assessed, and neither was its therapeutic potential for the treatment of chronic wounds.

### SUMMARY OF THE INVENTION

The subject invention provides a method for promoting wound healing in a patient in need thereof comprising contacting a wound of the patient with exosomes secreted by umbilical cord blood mononuclear cells (UCBMNCs) so as to thereby promote wound healing in the patient.

The subject invention also provides a composition comprising exosomes secreted by umbilical cord blood mononuclear cells (UCBMNCs) for promoting wound healing in a patient in need thereof.

The subject invention also provides a process of making a composition comprising exosomes secreted by umbilical cord blood mononuclear cells (UCBMNCs) comprising step a) separating the exosomes from the UCBMNCs and b) suspending the exosomes in a pharmaceutically acceptable carrier.

The subject invention also provides a wound care product comprising exosomes secreted by umbilical cord blood mononuclear cells (UCBMNCs). Preferably, the exosomes contain miRNA hsa-miR-150-5p.

The subject invention also provides a method for promoting wound healing in a patient in need thereof comprising contacting a wound of the patient with a composition comprising miRNA hsa-miR-150-5p so as to thereby promote wound healing in the patient.

The subject invention also provides a composition comprising miRNA hsa-miR-150-5p and a pharmaceutically acceptable carrier for promoting wound healing in a patient in need thereof.

The subject invention also provides a process of making a composition comprising miRNA hsa-miR-150-5p and a pharmaceutically acceptable carrier comprising step a) mixing the miRNA hsa-miR-150-5p with a transfection agent, and b) suspending the mixture in a pharmaceutically acceptable carrier.

### BRIEF DESCRIPTION OF THE DRAWINGS

FIGS. 1A-1B. Characterization of Umbilical Cord Blood Mononuclear Cells and the impact of cryopreservation, as analysed in the haematological analyser.

In FIG. 1A Detailed composition of the different white blood cells subpopulations present in umbilical cord blood (lymphocytes, monocytes, neutrophils, eosinophils and basophils).

In FIG. 1B Impact of processing in the composition of umbilical cord blood mononuclear cells (total lymphocytes and monocytes) relative to the total white blood cells.

FIGS. 2A-3E. Characterization of exosomes secreted by Umbilical Cord Blood Mononuclear Cells.

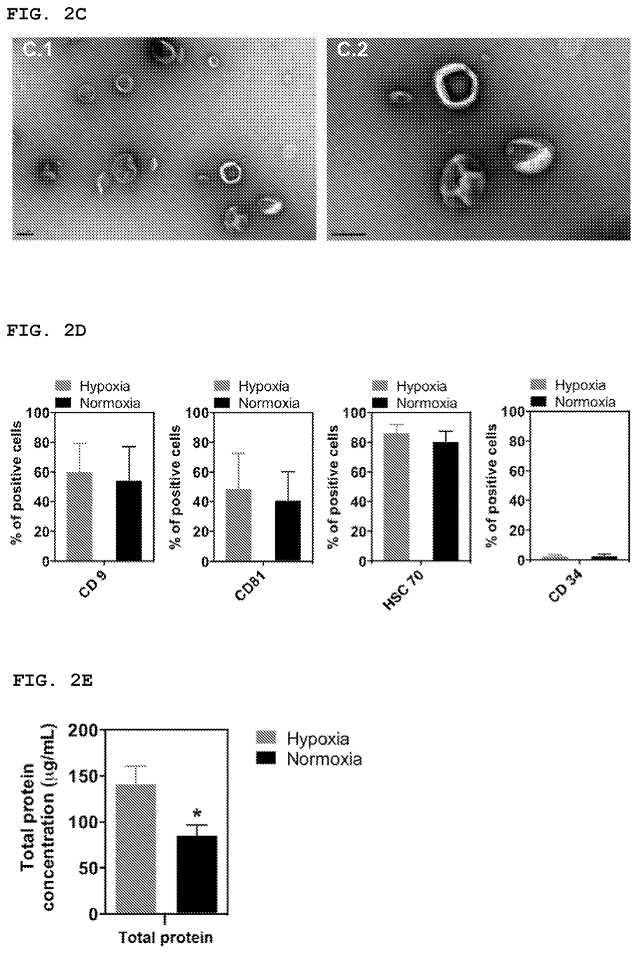

In FIG. 2A Size distribution of exosomes by DLS.

In FIG. 2B Zeta potential of exosomes as evaluated by DLS.

In FIG. 2C Size and morphology of exosomes secreted by UCBMNCs as evaluated by TEM. Size and morphology of exosomes secreted by UCBMNCs as evaluated by TEM.

In FIG. 2D Expression of surface markers in exosomes secreted by UCBMNCs preconditioned in hypoxia and in control conditions (normoxia) as analyzed by flow cytometry. Results are average+/−SEM, n=3.

In FIG. 2E Total protein content, as quantified with the DC protein assay from Biorad of exosomes secreted by UCBMNCs preconditioned in hypoxia (Exo-Hyp) and normoxia (Exo_Nor). Results are average+/−SEM, n=3. Statistical analyses were performed by a t-test. *p<0.05.

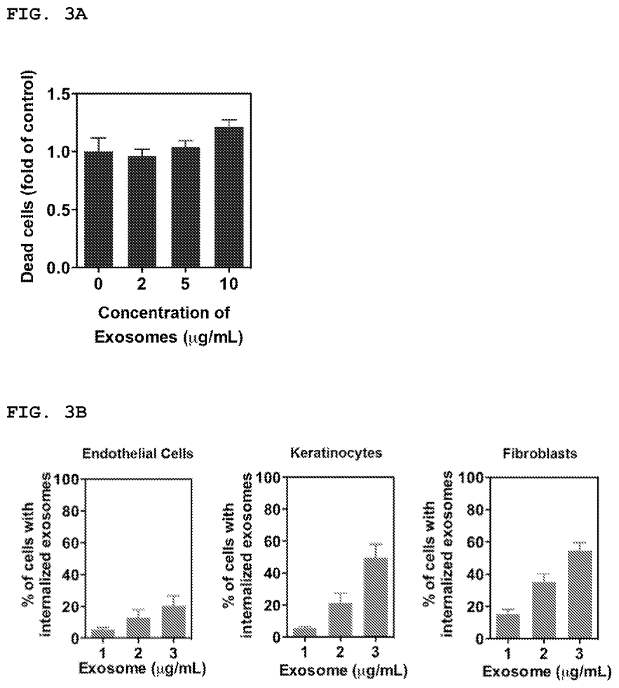

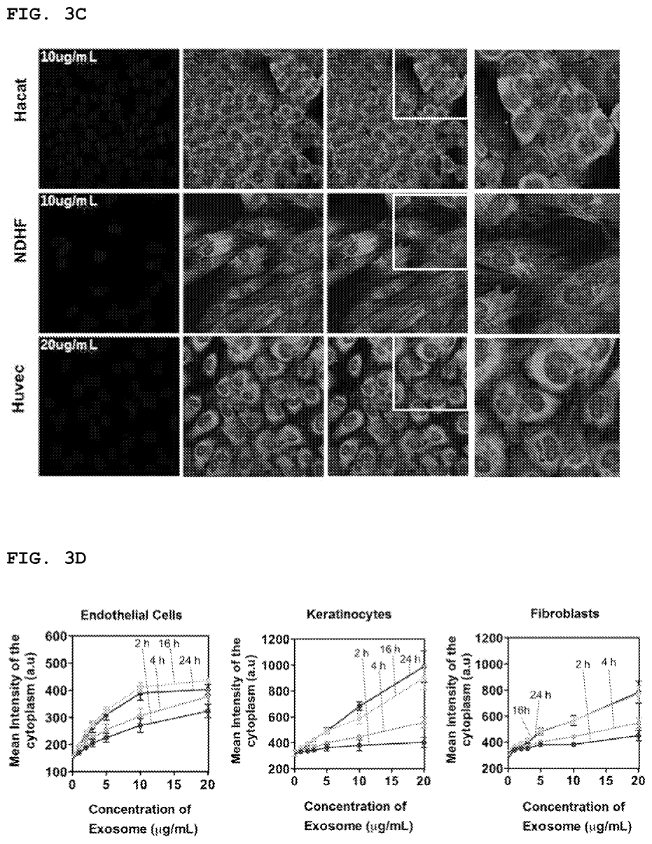

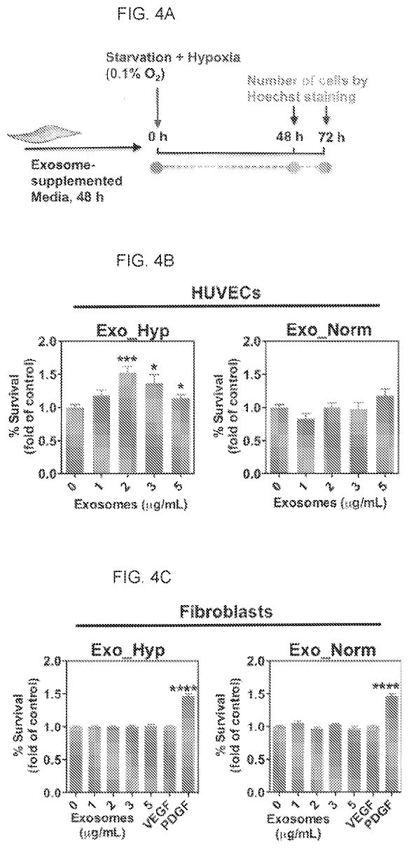

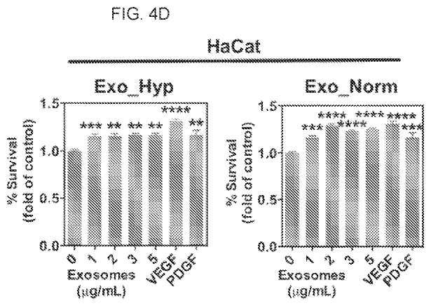

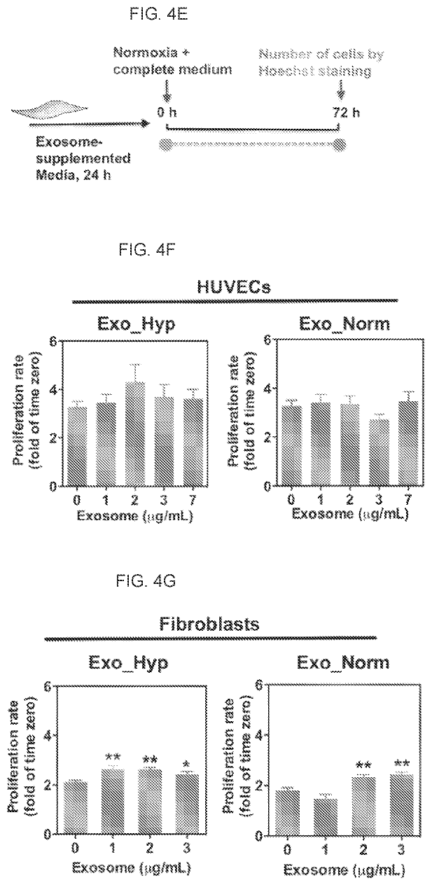

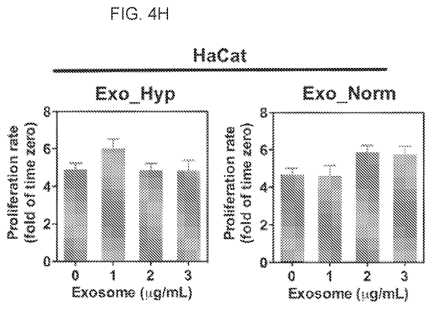

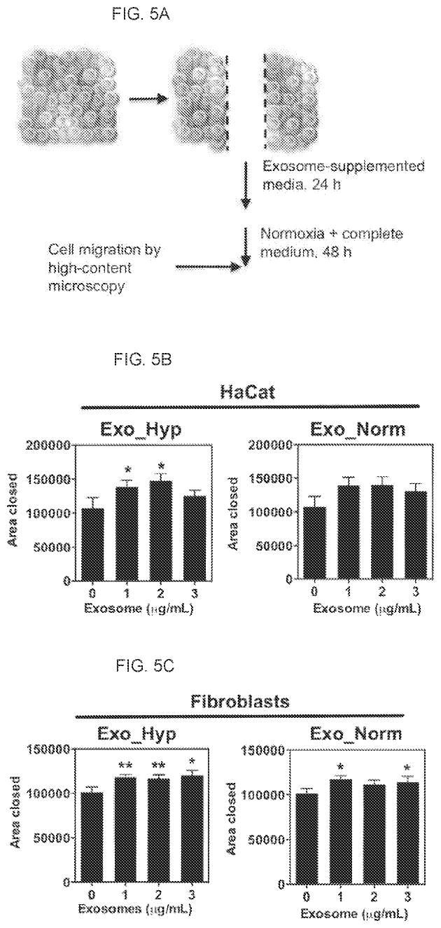

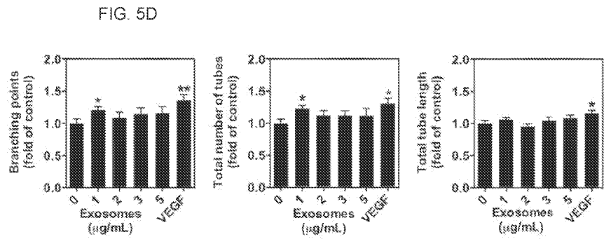

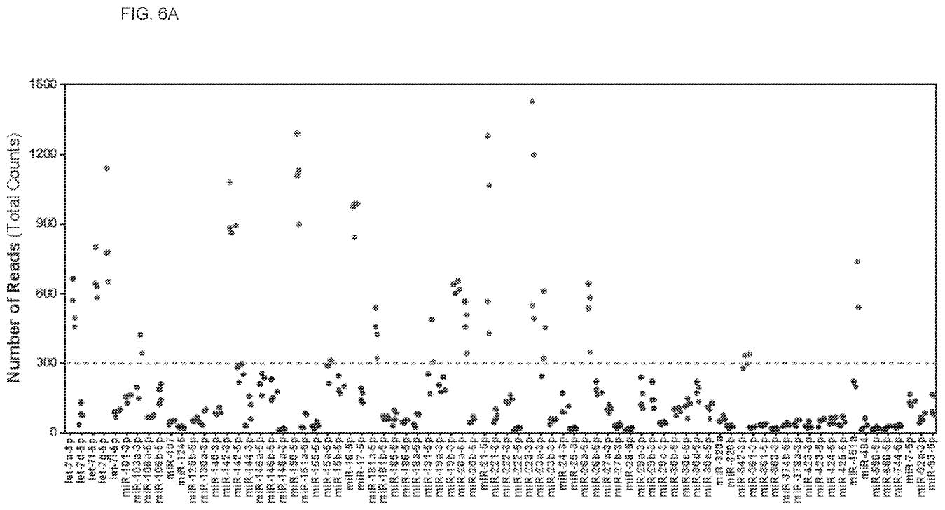

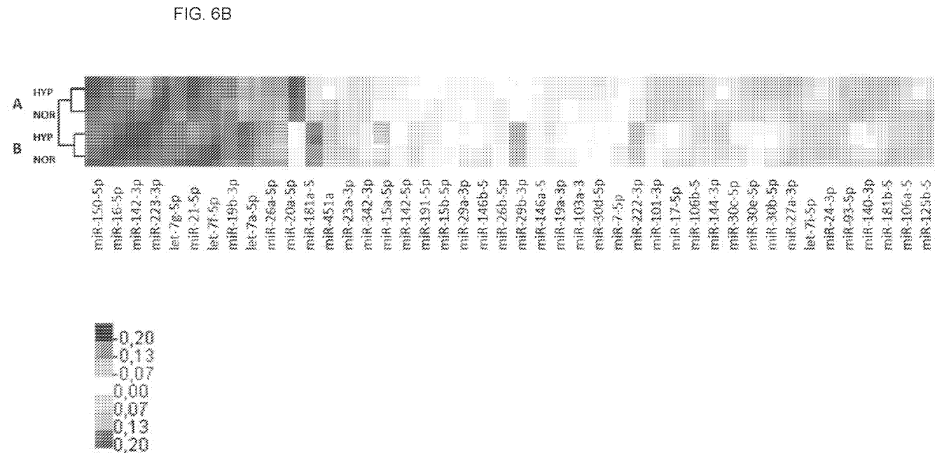

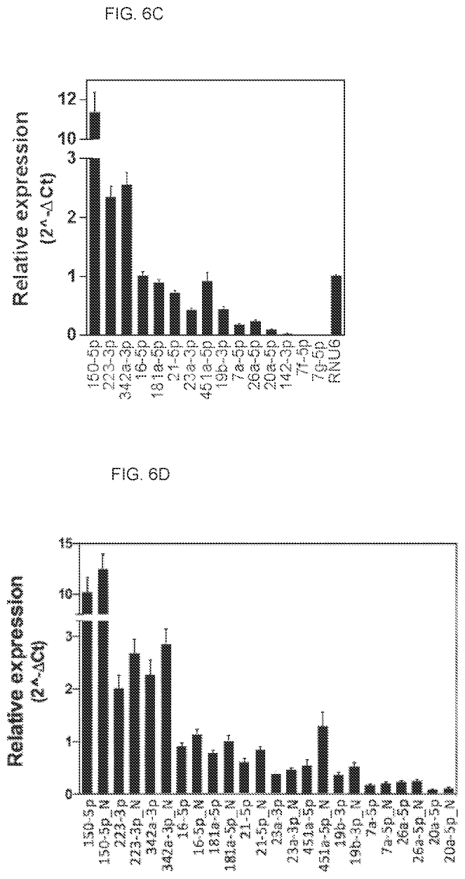

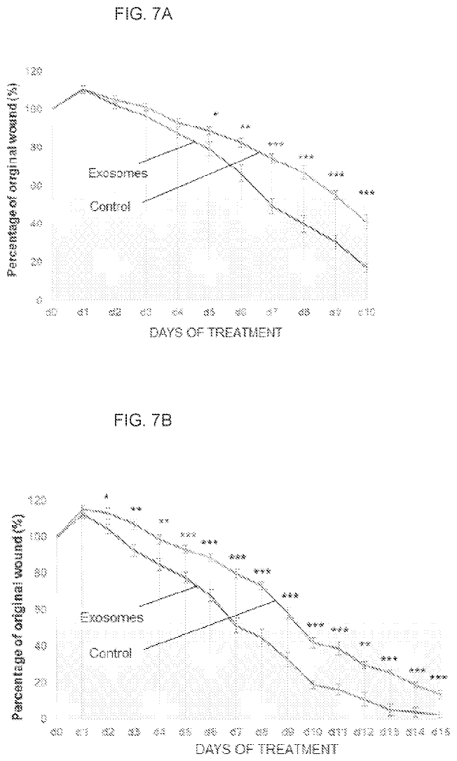

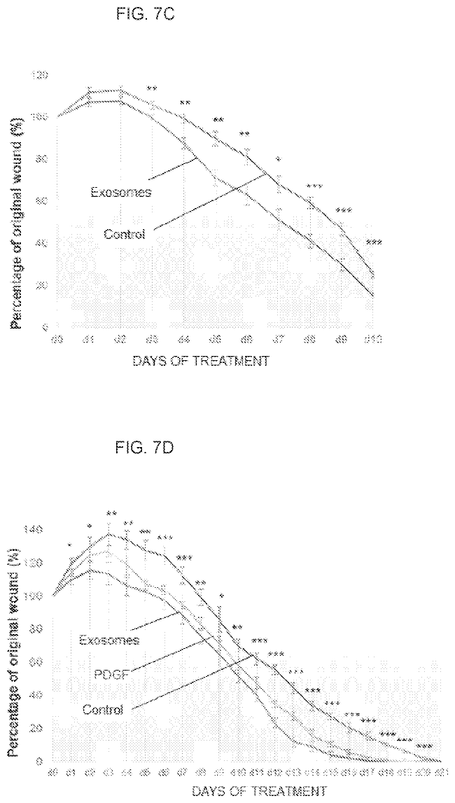

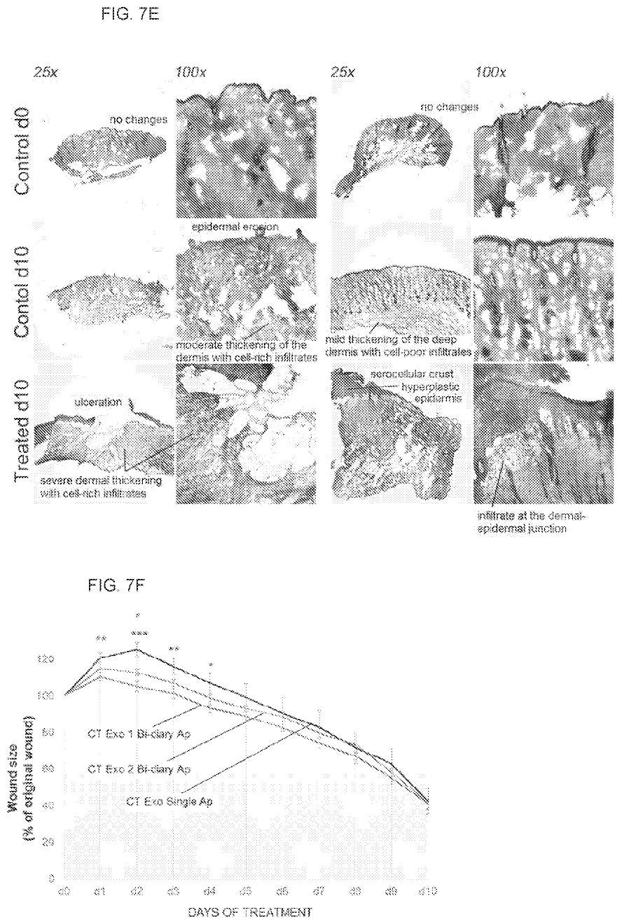

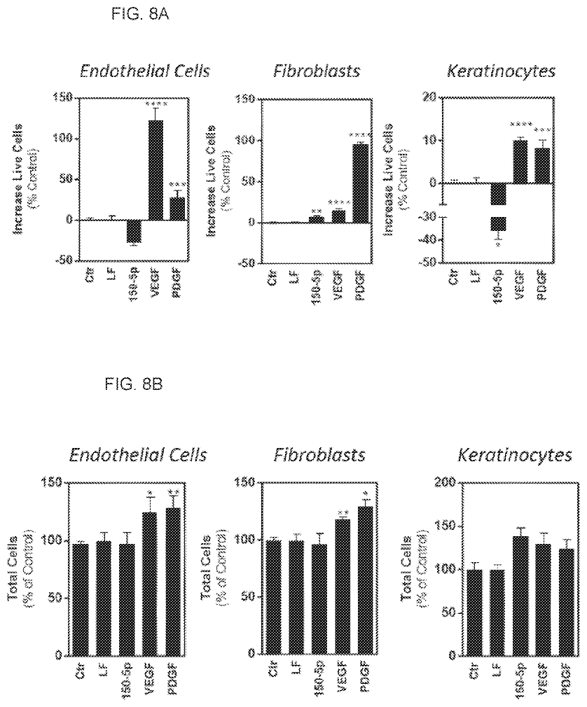

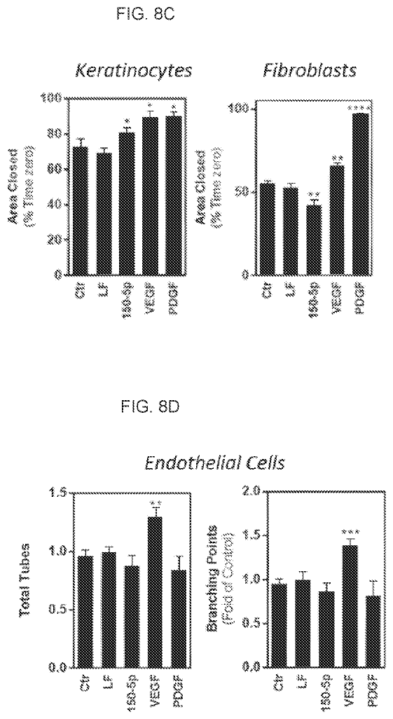

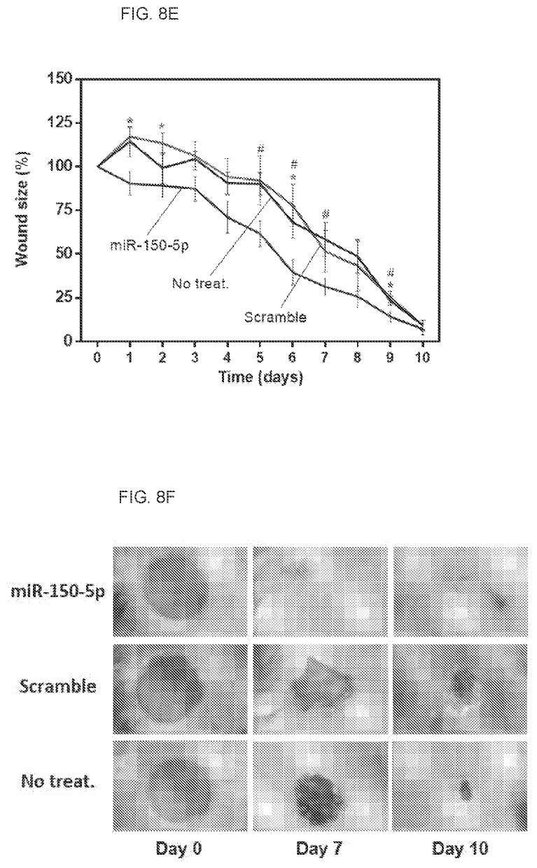

FIGS. 3A-3D. Exosome cytotoxicity and exosome uptake in endothelial cells, fibroblasts and keratinocytes.

In FIG. 3A Cytotoxicity of exosomes (from 3 different donors) in HUVECs as evaluated by propidium iodide/Hoechst staining. Cells were exposed to exosomes for 24 h and viability assessed after 72 h. Results are average+/−SEM, n=3 per donor. Approximately 2000 cells were quantified in each image.

In FIG. 3BUptake of exosomes by endothelial cells, fibroblasts and keratinocytes, as evaluated by flow cytometry. Results are average +/−SEM, n=4-6.

In FIG. 3C Confocal microscopy of skin cells exposed to Syto® RNASelect™ labeled exosomes (1-20 μg/ml) in exo-depleted medium and incubated for 16 h.

In FIG. 3D Quantification of Syto® RNASelectm labeled exosomes in HUVECs, HaCaT cells and fibroblasts. Results are average+/−SEM, n=4.

FIGS. 4A-4H. Bioactivity UCBMNCs-Exo in skin cells. In FIGS. 4A-4D Exosomes secreted by CD34-depleted UCBMNC cells subjected to ischemic pre-conditioning (Exo_Hyp) increase the survival of endothelial cells and keratinocytes but not fibroblasts. Results were obtained for exosomes collected from 3 different donors. The results are average+/−SEM, n=3 per donor. Vascular endothelial growth factor (VEGF, 50 ng/mL) or platelet derived growth factor (PDGF-BB, 50 ng/mL) were used as controls.

In FIGS. 4E-4H Proliferation of endothelial cells, fibroblasts and keratinocytes upon treatment with Exo_Hyp or Exo_Nor. Results were obtained for exosomes collected from 3 different donors. The results are average+/−SEM, n=3 per donor.

*p<0.05; **p<0.01; ***p<0.001.

FIGS. 5A-5D. Bioactivity UCBMNCs-Exo in skin cells.

In FIGS. 5A-5C Scratch assay in keratinocytes and fibroblasts after treatment with Exo_Hyp or Exo_Nor.

In FIG. 5D Endothelial tube assay in Matrigel. HUVECs were treated with Exo_Hyp or Exo_Nor.

In (A) and (B), results were obtained for exosomes collected from 3 different donors. The results are average+/−SEM, n=3 per donor.

*p<0.05; **p<0.01; ***p<0.001.

FIGS. 6A-6D. Characterization of UCBMNC-Exo miRNA profile shoiwng

In FIGS. 6A-6B miRNAs identified in Exo_Hyp and Exo_Nor in two different donors. In FIG. 6A 69 different miRNAs were identified in exosomes secreted by hUCBMNC CD34-depleted from two different donors (A and B) subjected to hypoxia (HYP) and control conditions (NOR) by RNASeq, counting more than 20 reads in all exosomes analyzed.

In FIG. 6B Treeview representation of 44 miRs with more than 50 reads for exosomes obtained after conditioning hUCBMNCs of both donors in hypoxia (HYP) and normoxia (NOR).

In FIGS. 6C-6D Average expression of miRNAs for 10 donors. In FIG. 6C Relative expression of the 15 most expressed miRNAs, detected by RNASeq, of exosomes from hypoxia conditioned hUCBMNCs. RNU6 was used as control. Results were obtained for exosomes collected from 10 different donors. Results are average+/−SEM, n=3-4 per donor.

In FIG. 6D Relative expression of 12 most expressed miRNAs of exosomes from hypoxia (HYP) vs normoxia (NOR) conditioned hUCBMNCs. RNU6 was used as control. Results were obtained for exosomes collected from 10 different donors. Results are average+/−SEM, n=3-4 per donor. Statistical analysis was performed by one-way ANOVA with Newman-Keuls multiple comparisons test, *p<0.05; **p<0.01; ***p<0.001.

FIGS. 7A-7F. UCBMNC-Exo stimulate wound healing in normal and Type I and II diabetic mouse models.

In FIG. 7AUCBMNC-Exo from Donor 1; In FIG. 7B UCBMNC-Exo from Donor 2; In FIG. 7C UCBMNC-Exo from Donor 1 administered to non-diabetic animals; In FIG. 7D UCBMNC-Exo from Donor 1 administered to genetic diabetic animals (db/db);

In FIG. 7E H&E staining of control and treated wounds (diabetes Type 1) at day 10;

In FIG. 7F Comparison of the wound closure rates between wounds treated with control saline solution (Control Wound), when UCBMNC-Exo are applied at a distant wound only once on day 1 (CT Exo Single Ap), or twice daily (CT Exo 1 Bi-diary Ap and CT Exo 2 Bi-diary Ap). UCBMNC-Exo applied twice daily to distant excisional wounds stimulate wound healing at the control wound.

FIGS. 8A-8E. MiR-150-5p enhances fibroblasts survival to ischemia and keratinocyte migration in vitro, and wound healing in vivo.

In FIGS. 8A-8D In vitro effect of miR-150-5p in FIG. 8A Survival in ischemic conditions and =In FIG. 8B Proliferation of endothelial cells, fibroblasts and keratinocytes, In FIG. 8C) Scratch assay of fibroblasts and keratinocytes and In FIG. 8D tube formation of endothelial cells in Matrigel. Vascular endothelial growth factor (VEGF, 50 ng/mL) and platelet derived growth factor (PDGF-BB, 50 ng/mL) were used as controls for all the assays. The results are average+/−SEM, n=3-6. Statistical analyses were performed by Mann-Whitney test, *p<0.05; **p<0.01; ***p<0.001.

In FIG. 8E In vivo effect of miR-150 in wound healing of excisional wounds inflicted in C57Bl/6. The results are average+/−SEM, n=5-6. Statistical analyses were performed by t-test, p<0.05 for Scramble treatment (*); for ‘No treat.’=PBS (#). In FIG. 8F Representative images at day 0; 7 and 10 of the wounds treated with miR-150-5p, Scramble and PBS (No treat.).

### DETAILED DESCRIPTION OF THE INVENTION

The subject invention provides a method for promoting wound healing in a patient in need thereof comprising contacting a wound of the patient with a composition comprising exosomes secreted by umbilical cord blood mononuclear cells (UCBMNCs) so as to thereby promote wound healing in the patient.

In one embodiment, the composition further comprises a pharmaceutically acceptable carrier.

In one embodiment, the UCBMNCs comprise lymphocytes, monocytes, neutrophils, eosinophils, and/or basophils.

In one embodiment, the UCBMNCs comprise lymphocytes. In one embodiment, the UCBMNCs consists of lymphocytes. In one embodiment, the UCBMNCs consists essentially of lymphocytes.

In one embodiment, the UCBMNCs comprise monocytes. In one embodiment, the UCBMNCs consists of monocytes. In one embodiment, the UCBMNCs consists essentially of monocytes.

In one embodiment, the UCBMNCs comprise neutrophils. In one embodiment, the UCBMNCs comprise eosinophils. In one embodiment, the UCBMNCs comprise basophils.

In one embodiment, the UCBMNCs comprise lymphocytes and monocytes. In one embodiment, the UCBMNCs consists essentially of lymphocytes and monocytes. In one embodiment, the UCBMNCs consists of lymphocytes and monocytes.

In one embodiment, the UCBMNCs comprise lymphocytes and neutrophils. In one embodiment, the UCBMNCs comprise monocytes and neutrophils.

In one embodiment, the UCBMNCs comprise CD34+ cells. In another embodiment, the UCBMNCs do not comprise CD34+ cells.

In one embodiment, the UCBMNCs were not exposed to ischemic conditions. In another embodiment, the UCBMNCs were exposed to ischemic conditions. In one embodiment, the UCBMNCs were exposed to hypoxic conditions. In one embodiment, the UBCMNCs were not exposed to hypoxic conditions.

In one embodiment, exposure to ischemic and/or hypoxic conditions stimulates increased secretion of exosomes from UCBMNCs. In another embodiment, exposure to ischemic and/or hypoxic conditions increased bioactivity of the exosomes secreted. In one embodiment, the bioactivity is pro-survival bioactivity. In another embodiment, the bioactivity is pro-proliferation bioactivity. In another embodiment, the bioactivity is pro-migration bioactivity.

In one embodiment, at least one of the exosomes exhibit a spherical morphology.

In one embodiment, the exosomes have an average particle size of 100 nm to 200 nm. In one embodiment, the exosomes have an average particle size of 120 nm to 170 nm. In one embodiment, the exosomes have an average particle size of 130 nm to 150 nm. In one embodiment, the exosomes have an average particle size of 140 nm.

In one embodiment, the exosomes have a negative zeta potential of −10 mV to −50 mV. In one embodiment, the exosomes have a negative zeta potential of −20 mV to −40 mV. In one embodiment, the exosomes have a negative zeta potential of −30 mV.

In one embodiment, at least one of the exosomes express the markers CD9, CD34, CD81, and/or Hsc70.

Claim 7.1—In one embodiment, at least one of the exosomes contain one or more miRNA selected from a group consisting of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

7.2 In one embodiment, at least one of the exosomes contain one or more miRNA selected from a group consisting of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, and hsa-miR-146b-5p.

7.3 In one embodiment, at least one of the exosomes contain one or more miRNA selected from a group consisting of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, and hsa-miR-146a-5p.

7.4 In one embodiment, at least one of the exosomes contain one or more miRNA selected from a group consisting of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, and hsa-miR-342-3p.

7.5 In one embodiment, at least one of the exosomes contain one or more miRNA selected from a group consisting of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, and hsa-miR-26a-1-5p.

7.6 In one embodiment, at least one of the exosomes contain one or more miRNA selected from a group consisting of: hsa-miR-150-5p, hsa-miR-16-5p, hsa-miR-142-3p, hsa-miR-223-3p, hsa-let-7g-5p, and hsa-miR-21-5p.

7.7 In one embodiment, at least one of the exosomes contain one or more miRNA selected from a group consisting of: hsa-miR-181a-5p, hsa-miR-451a, hsa-miR-103a-3p, hsa-miR-15a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

In one embodiment, at least one of the exosomes contain the miRNA hsa-miR-150-5p. In one embodiment, at least 10% of the exosomes contain the miRNA hsa-miR-150-5p. In one embodiment, at least 25% of the exosomes contain the miRNA hsa-miR-150-5p. In one embodiment, at least 50% of the exosomes contain the miRNA hsa-miR-150-5p. In one embodiment, at least 75% of the exosomes contain the miRNA hsa-miR-150-5p. In one embodiment, at least 90% of the exosomes contain the miRNA hsa-miR-150-5p.

In one embodiment, the miRNA hsa-miR-150-5p has a higher concentration in the exosomes than the concentration of any other one miRNA in the exosomes.

In one embodiment, the exosomes are enriched in one or more miRNA selected from a group consisting of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p, thereby increasing the concentration of the miRNA in the exosomes.

In one embodiment, the exosomes are enriched in one or more miRNA selected from a group consisting of: hsa-miR-181a-5p, hsa-miR-451a, hsa-miR-103a-3p, hsa-miR-15a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p, thereby increasing the concentration of the miRNA in the exosomes.

In one embodiment, the exosomes are enriched in miRNA hsa-miR-150-5p, thereby increasing the concentration of the miRNA in the exosomes.

The exosomes may be enriched in miRNA using conventional methods known in the art including but not limited to differential centrifugation, density gradient/cushion centrifugation, size-exclusion chromatography, precipitation, immuno affinity capture on beads, and RNA Lading techniques such as techniques based on electroporation, virus infection of the secreting cells, and targeted embedding of specific RNA sequences via sequence-recognition domains fused to vesicular proteins.

In one embodiment, the composition is a liquid. In one embodiment, the composition is a cream. In one embodiment, the composition is a gel.

In one embodiment, the composition is administered in a single application. In another embodiment, the composition is administered periodically. In another embodiment, the composition is administered less often than once daily. In another embodiment, the composition is administered once daily. In another embodiment, the composition is administered more often than once daily. In another embodiment, the composition administered twice daily. In another embodiment, the composition administered three times daily.

In one embodiment, the composition is applied topically. In one embodiment, the composition is applied by subcutaneous injection. In one embodiment, the composition is applied by dermal injection. In one embodiment, the composition is applied by intraperitoneal injection. In one embodiment, the composition is applied by intravenous injection.

In one embodiment, the concentration of exosomes in the composition is 0.01 μg/mL-10 μg/mL. In one embodiment, the concentration of exosomes is 1 μg/mL-3 μg/mL. In one embodiment, the concentration of exosomes is 0.5 μg/mL-1.5 μg/mL. In one embodiment, the concentration of exosomes is about 1 μg/mL. In one embodiment, the concentration of exosomes is 0.01 μg/mL. In one embodiment, the concentration of exosomes is 0.25 μg/mL. In one embodiment, the concentration of exosomes is 0.5 μg/mL. In one embodiment, the concentration of exosomes is 0.75 μg/mL. In one embodiment, the concentration of exosomes is 1.0 μg/mL. In one embodiment, the concentration of exosomes is 1.25 μg/mL. In one embodiment, the concentration of exosomes is 1.5 μgg/mL. In one embodiment, the concentration of exosomes is 1.75 μg/mL. In one embodiment, the concentration of exosomes is 2.0 μg/mL. In one embodiment, the concentration of exosomes is 2.25 μg/mL. In one embodiment, the concentration of exosomes is 2.5 μg/mL. In one embodiment, the concentration of exosomes is 2.75 μg/mL. In one embodiment, the concentration of exosomes is 3.0 μg/mL. In one embodiment, the concentration of exosomes is 5.0 μg/mL. In one embodiment, the concentration of exosomes is 10 μg/mL.

In one embodiment, the composition further comprises one or more miRNA selected from a group consisting of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p, wherein the miRNA is not contained in the exosomes.

In one embodiment, the composition further comprises one or more miRNA selected from a group consisting of: hsa-miR-181a-5p, hsa-miR-451a, hsa-miR-103a-3p, hsa-miR-15a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p, wherein the miRNA is not contained in the exosomes.

In one embodiment, the composition further comprises miRNA hsa-miR-150-5p wherein the miRNA hsa-miR-150-5p is not contained in the exosomes.

In one embodiment, wound healing is promoted by accelerating the rate of wound healing. In one embodiment, wound healing is promoted by inducing vascularization.

In one embodiment, wound healing is promoted by increasing survival of cells. In one embodiment, wound healing is promoted by increasing survival of keratinocytes. In another embodiment, wound healing is promoted by stimulating survival of endothelial cells.

In one embodiment, wound healing is promoted by increasing proliferation of cells. In one embodiment, wound healing is promoted by increasing proliferation of fibroblasts.

In one embodiment, wound healing is promoted by increasing migration of cells towards the wound. In one embodiment, wound healing is promoted by increasing migration of fibroblasts toward the wound. In another embodiment, wound healing is promoted by increasing migration of keratinocytes toward the wound.

In one embodiment, wound healing is promoted by increasing tube-formation at and/or near the site of the wound. In one embodiment, wound healing is promoted by inducing tube-formation by endothelial cells.

In one embodiment, the exosomes are effective to increase the total number of vascular tubes formed. In another embodiment, the exosomes are effective to increase the complexity of the vascular network formed. In one embodiment, the complexity of the vascular network formed is assessed by the augmented number of branching points.

In one embodiment, the keratinocytes have been exposed to ischemic conditions. In one embodiment the keratinocytes have been exposed to hypoxic conditions. In one embodiment, the fibroblasts have been exposed to ischemic conditions. In one embodiment the fibroblasts have been exposed to hypoxic conditions. In one embodiment, the endothelial cells have been exposed to ischemic conditions. In one embodiment the endothelial cells have been exposed to hypoxic conditions.

In one embodiment, exosomes exposed to ischemic conditions are more effective in inducing proliferation of fibroblasts than exosomes not exposed to ischemic conditions.

In one embodiment, exosomes exposed to ischemic conditions are more effective in inducing proliferation of fibroblasts at a concentration of 1 μg/mL than exosomes not exposed to ischemic conditions at a concentration of 1 μg/mL. In one embodiment, exosomes exposed to ischemic conditions are more effective in inducing proliferation of fibroblasts at a concentration of 2 μg/mL than exosomes not exposed to ischemic conditions at a concentration of 2 μg/mL.

In another embodiment, exosomes exposed to hypoxic conditions are more effective in inducing proliferation of fibroblasts. In one embodiment, exosomes exposed to hypoxic conditions are more effective in inducing proliferation of fibroblasts at a concentration of 1 μg/mL. In one embodiment, exosomes exposed to hypoxic conditions are more effective in inducing proliferation of fibroblasts at a concentration of 2 μg/mL.

In one embodiment, wound healing is promoted by the miRNA hsa-miR-150-5p.

In another embodiment, the miRNA hsa-miR-150-5p increases migration of keratinocytes towards the wound thereby promoting wound healing.

In another embodiment, the miRNA hsa-miR-150-5p increases survival of fibroblast survival thereby promoting wound healing.

In one embodiment, wound healing is promoted at a site local to the site of contact of the exosomes. In another embodiment, wound healing is promoted at a site distant from the site of contact of the exosomes.

In another embodiment, the wound is a chronic wound.

In one embodiment, the wound is a pressure ulcer. In another embodiment, the wound is a venous ulcer. In another embodiment, the wound is a post-surgical ulcer. In another embodiment, the wound is a traumatic ulcer. In another embodiment, the wound is a lower extremity wound. In another embodiment, the wound is an arterial ulcer.

In one embodiment, the wound is a mouth ulcer.

In one embodiment, the wound is an eye wound or a corneal ulcer.

In one embodiment, the patient is afflicted with diabetes. In one embodiment, the patient is not afflicted with diabetes.

In another embodiment, the patient is afflicted with type I diabetes. In another embodiment, the patient is afflicted with type II diabetes.

In one embodiment, the wound is a diabetic ulcer. In one embodiment, the diabetic ulcer is a diabetic foot ulcer.

In one embodiment, the wound is an open wound. In one embodiment, the open wound is an incision. In one embodiment, the open wound is a laceration. In one embodiment, the open wound is a tear. In one embodiment, the open wound is an abrasion. In one embodiment, the open wound is an avulsion. In one embodiment, the open wound is a puncture.

In one embodiment, the wound is an excisional wound. In another embodiment, the wound is an infectious wound. In another embodiment, the wound is an ischemic wound. In another embodiment, the wound is a radiation-poisoning wound. In another embodiment, the wound is a surgical wound.

In one embodiment, the wound is a burn. In one embodiment, the burn is a thermal burn. In one embodiment, the burn is a chemical burn. In one embodiment, the burn is a radiation burn.

In one embodiment, the wound relates to a skin condition or disease comprising a wound such as acne, psoriasis, rosacea, dermatitis, eczema, impetigo, intertrigo, or folliculitis.

In one embodiment, the accelerated rate of wound healing is significant by day 2. In another embodiment, the accelerated rate of wound healing is significant by day 3. In another embodiment, the accelerated rate of wound healing is significant by day 10.

In one embodiment, wound healing is assessed by wound closure. In one embodiment, wound closure is improved by at least 10%. In another embodiment, wound closure is improved by at least 20%. In another embodiment, wound closure is improved by at least 30%. In another embodiment, wound closure is improved by at least 50%. In another embodiment, wound closure is improved by at least 80%. In another embodiment, wound closure is improved by more than 100%.

In one embodiment, the time taken for complete wound closure is reduced by at least 10%. In another embodiment, the time taken for complete wound closure is reduced by at least 20%. In another embodiment, the time taken for complete wound closure is reduced by at least 30%. In another embodiment, the time taken for complete wound closure is reduced by at least 50%. In another embodiment, the time taken for complete wound closure is reduced by at least 80%. In another embodiment, the time taken for complete wound closure is reduced by more than 100%.

The subject invention provides a composition comprising exosomes secreted by umbilical cord blood mononuclear cells (UCBMNCs).

The subject invention provides a composition comprising exosomes secreted by umbilical cord blood mononuclear cells (UCBMNCs) for use in promoting wound healing in a patient in need thereof.

The subject invention provides a composition comprising exosomes containing one or more miRNA selected from a group consisting of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

In one embodiment, the exosomes are secreted by umbilical cord blood mononuclear cells (UCBMNCs).

In one embodiment, the exosomes contain miRNA hsa-miR-150-5p.

The subject invention provides a process of making a composition comprising exosomes secreted by umbilical cord blood mononuclear cells (UCBMNCs) comprising step a) separating the exosomes from the UCBMNCs and b) suspending the exosomes in a pharmaceutically acceptable carrier.

In one embodiment, the pharmaceutically acceptable carrier is phosphate buffered saline (PBS). In one embodiment, the pharmaceutically acceptable carrier is a liquid suspension. In one embodiment, the pharmaceutically acceptable carrier is a polymer. In one embodiment, the pharmaceutically acceptable carrier is a hydrogel. In one embodiment, the pharmaceutically acceptable carrier is protein matrix. In one embodiment, the pharmaceutically acceptable carrier is a liposomal suspension. In one embodiment, the pharmaceutically acceptable carrier is a foam.

In one embodiment, the exosomes are separated from the UCBMNCs by centrifugation. In one embodiment, the exosomes are separated from the UCBMNCs by differential centrifugation.

The most commonly used technique to purify exosomes is differential ultracentrifugation, but other techniques may also be used for the subject invention including but not limited to ultracentrifugation in a density gradient, high-pressure liquid chromatography-gel exclusion chromatography (HPLC-GEC), solvent precipitation, ultrafiltration, immunoaffinity capture (IAC), and field flow fractionation (FFF).

Solvent precipitation is a technique that successfully separates exosomes at relatively slow spin speeds without an ultracentrifuge. Three examples of commercial kits using solvent precipitation are Exosome Isolation kit (Life Technologies), ExoQuick (System Bioscience), and Exo-spins (Cell Guidance System).

IAC relies on the binding of an antibody to its specific antigen. As the knowledge of the subcomponents of exosomes grows, more individual antigen targets (typically proteins) are identified that can be used to specifically identify exosomes.

FFF is a technique where particles are separated by their position in a laminar velocity gradient. In asymmetrical flow field flow fractionation, a cross-flow perpendicular to the down-channel flow drives particles toward the membrane that bounds the channel. Diffusion causes particles to migrate away from the membrane, opposing the cross-flow.

In one embodiment, the process further comprises a step of enriching the exosomes in one or more miRNA prior to step b), wherein the miRNA is selected from a group consisting of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

In one embodiment, the process further comprises a step of enriching the exosomes in one or more miRNA prior to step b), wherein the miRNA is selected from a group consisting of: hsa-miR-181a-5p, hsa-miR-451a, hsa-miR-103a-3p, hsa-miR-15a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

In one embodiment, the process further comprises a step of enriching the exosomes in miRNA hsa-miR-150-5p prior to step b).

In one embodiment, the composition further comprises one or more miRNA not contained in the exosomes and the process further comprises the steps of mixing the miRNA with a transfection agent and suspending the mixture in the pharmaceutically acceptable carrier, wherein the miRNA is selected from a group consisting of hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

In one embodiment, the miRNA is selected from a group consisting of hsa-miR-181a-5p, hsa-miR-451a, hsa-miR-103a-3p, hsa-miR-15a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

In one embodiment, the miRNA is hsa-miR-150-5p.

The subject invention also provides a composition comprising exosomes secreted by umbilical cord blood mononuclear cells (UCBMNCs) prepared by the processes described herein.

The subject invention also provides a wound care product comprising exosome secreted by umbilical cord blood mononuclear cells (UCBMNCs).

The subject invention provides a method for promoting wound healing in a patient in need thereof comprising contacting a wound of the patient with a composition comprising one or more miRNA selected from a group consisting of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

The subject invention provides a method for promoting wound healing in a patient in need thereof comprising contacting a wound of the patient with a composition comprising one or more miRNA selected from a group consisting of: hsa-miR-150-5p, hsa-miR-181a-5p, hsa-miR-451a, hsa-miR-103a-3p, hsa-miR-15a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

The subject invention provides a method for promoting wound healing in a patient in need thereof comprising contacting a wound of the patient with a composition comprising one or more miRNA selected from a group consisting of: hsa-miR-181a-5p, hsa-miR-451a, hsa-miR-103a-3p, hsa-miR-15a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

The subject invention provides a method for promoting wound healing in a patient in need thereof comprising contacting a wound of the patient with a composition comprising miRNA hsa-miR-150-5p so as to thereby promote wound healing in the patient.

In one embodiment, the amount of miRNA hsa-miR-150-5p applied is between 100 pmol to 1 nmol. In one embodiment, the amount of miRNA hsa-miR-150-5p applied is between 100 pmol to 800 pmol. In one embodiment, the amount of miRNA hsa-miR-150-5p applied is between 300 pmol to 500 pmol. In one embodiment, the amount of miRNA hsa-miR-150-5p applied is 400 pmol.

The subject invention provides a composition comprising miRNA selected from a group consisting of: hsa-miR-150-5p, hsa-miR-181a-5p, hsa-miR-451a, hsa-miR-103a-3p, hsa-miR-15a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p for promoting wound healing in a patient in need thereof.

In one embodiment, one of the miRNA is hsa-miR-150-5p.

In one embodiment, the composition further comprises a pharmaceutically acceptable carrier.

The subject invention provides a process of making a composition comprising one or more miRNA and a pharmaceutically acceptable carrier comprising step a) mixing the miRNA with a transfection agent, and b) suspending the mixture in a pharmaceutically acceptable carrier, wherein the miRNA is selected from a group consisting of: hsa-miR-150-5p, hsa-miR-181a-5p, hsa-miR-451a, hsa-miR-103a-3p, hsa-miR-15a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

In one embodiment, one of the miRNA is hsa-miR-150-5p.

The subject invention also provides a composition comprising miRNA and a pharmaceutically acceptable carrier prepared by the processes described herein.

The subject invention also provides a wound care product comprising the compositions described herein.

In one embodiment, the wound care product is a dressing or a pharmaceutical preparation.

In one embodiment, the dressing or pharmaceutical preparation is a graft material, patch, pad, plaster, film, tape, adhesive, gel, foam, cream, salve, balm, embrocation, ointment, poultice, unction, emollient, liniment, potion, unguent, lotion, spray, suspension, powder, syringe, nebuliser or aerosol container.

In one embodiment, the wound care product is:
a) a graft material comprising at least one fibroblast layer;
b) a gel or graft material comprising a fibrin matrix and fibroblasts;
c) a graft material comprising a matrix of at least one structural protein;
d) a graft material comprising a matrix of cross-linked collagen and glycosaminoglycan;
e) a gel, patch, pad, plaster, film or adhesive comprising a matrix of hydrophilic polymers dispersed in water;
f) a polyurethane film and/or a foam;
g) a pharmaceutical preparation comprising extracellular matrix protein, alginate and water; or
h) an aerosol container comprising an aerosol comprising said exosomes.

In one embodiment, the wound care product further comprises an antiseptic or antibacterial agent.

The subject invention also provides a sealed package comprising the compositions described herein.

The subject invention also provides a method for promoting wound healing in a patient in need thereof comprising contacting a wound of the patient with a composition as described herein so as to thereby promoting wound healing in the patient.

The subject invention also provides use of a composition as described herein for promoting wound healing in a patient in need thereof.

The subject invention also provides use of a composition as described herein in the manufacturing of a medicament for promoting wound healing in a patient in need thereof.

The subject invention provides a method for promoting tissue repair in a patient in need thereof comprising contacting a tissue of the patient with exosomes secreted by umbilical cord blood mononuclear cells (UCBMNCs) so as to thereby promote tissue repair in the patient.

The subject application also provides a method for promoting tissue repair in a patient in need thereof comprising contacting a tissue of the patient with a composition comprising one or more miRNA selected from a group consisting of: hsa-miR-181a-5p, hsa-miR-451a, hsa-miR-103a-3p, hsa-miR-15a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

The subject invention provides a method for promoting tissue repair in a patient in need thereof comprising contacting a tissue of the patient with a composition comprising miRNA hsa-miR-150-5p so as to thereby promote tissue repair in the patient.

Each embodiment disclosed herein is contemplated as being applicable to each of the other disclosed embodiments. Thus, all combinations of the various elements described herein are within the scope of the invention.

### Terms

Unless otherwise defined, all technical and scientific terms used herein have the same meaning as commonly understood by a person of ordinary skill in the art to which this invention belongs.

As used herein, and unless stated otherwise or required otherwise by context, each of the following terms shall have the definition set forth below.

The term “wound” as used herein refers to an injury to any living tissue. A wound may be caused by an act, by an infectious disease, or by an underlying condition. A wound may be chronic or acute. Examples of wound include but is not limited to cuts, ulcers, diabetic ulcers, ischemic tissue damage, ischemic heart damage, etc.

All publications and other references mentioned herein are incorporated by reference in their entirety, as if each individual publication or reference were specifically and individually indicated to be incorporated by reference. Publications and references cited herein are not admitted to be prior art.

This invention will be better understood by reference to the Experimental Details which follow, but those skilled in the art will readily appreciate that the specific experiments detailed are only illustrative of the invention as defined in the claims which follow thereafter.

### EXPERIMENTAL DETAILS

### Example 1. Ischemic Preconditioning Stimulates Umbilical Cord Blood Mononuclear Cells (UCBMNCs) to Secrete Exosomes and Increases their Bioactivity Towards Skin Cells

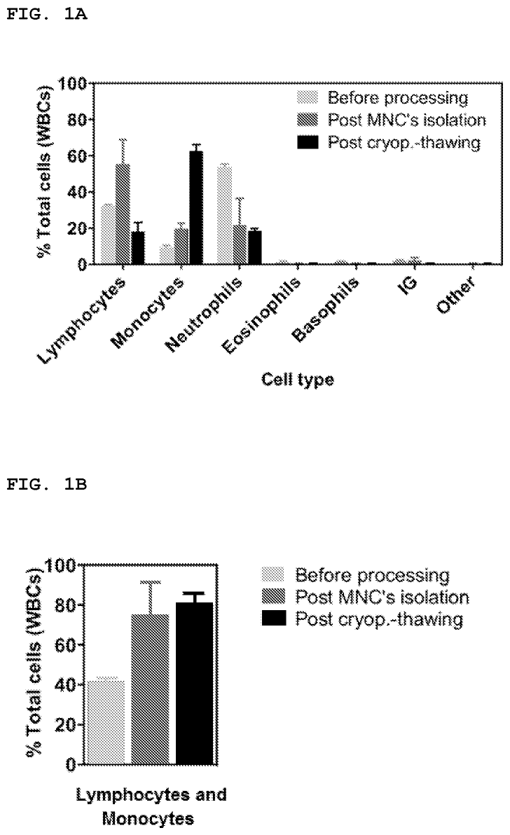

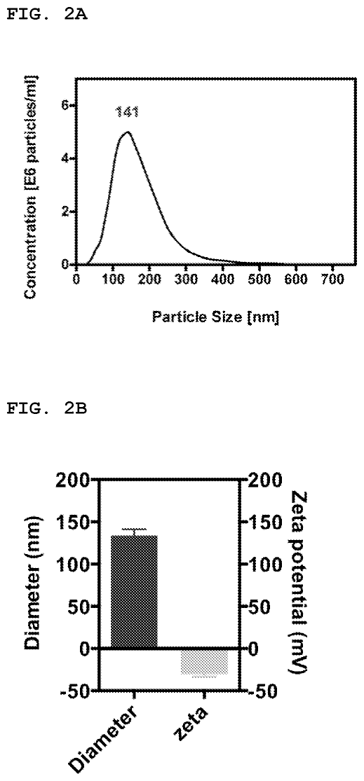

The total fraction of UCBMNCs was isolated using Lymphoprepm and the cells were characterized using a hematological analyzer. After cryopreservation, the population is enriched in lymphocytes and monocytes (MNC), while a significant fraction of neutrophils are lost (FIGS. 1A and 1B). The pool used for secretion of exosomes is mainly composed of lymphocytes and monocytes.

The experiments demonstrated that ischemic pre-conditioning (0.5% O₂, 5% CO₂, 0% FBS, 18 h) stimulates UCBMNCs to secrete bioactive exosomes which are internalized with specific kinetics by different types of skin cells (fibroblasts, keratinocytes and endothelial cells). The experiments also demonstrated that these exosomes (UCBMNCs-Exo) exert a potent pro-regenerative effect in vitro, which involves increasing the survival and proliferation of skin cells (fibroblasts, keratinocytes, and endothelial cells), as well as stimulating migration of fibroblasts and keratinocytes and tube formation by endothelial cells. Furthermore, the experiments demonstrated that topical administration of UCBMNCs-Exo twice a day significantly accelerates the healing of excisional wounds in normal and in Type 1 and 2 diabetic animal models.

### Example 1.1: Characterization of Exosomes

### Experimental Procedure

Exosomes were purified by differential centrifugation as described in Thery C. et al. (2006), the entire content of which is hereby incorporated.

Briefly, the supernatant was subjected to sequential centrifugation steps of 300g (10 minutes), 2000g (20 minutes), and two times at 10,000g (30 minutes), for removal of cells, cell debris, and microvesicles, respectively. After a 2 h centrifugation at 100000g in a SW41Ti or SW32Ti swinging bucket rotors (Beckman) the pellet was washed with 7 mL of PBS and centrifuged at 100000g for another 2 h. The pellet containing the exosomes was re-suspended in 250 uL of PBS. The exosomes were quantified based in their protein content using a DC Protein assay Kit from BioRad™.

The exosomes secreted by UCBMNC were initially characterized using transmission electron microscopy (TEM) and dynamic light scattering (DLS). The DLS analyses were performed in a Zeta PALS Zeta Potential Analyzer, containing a ZetaPlus Particle Sizing Software, v. 2.27 (Brookhaven Instruments Corporation). For particle size, exosomes (50 μL) were diluted in PBS (200 μL) and five measurements per sample were acquired at 25° C. for n=17. For the zeta potential, exosomes (50 μL) were diluted 25 times with biological molecular grade water filtered through a 0.2 μm pore (final PBS concentration of 5 mM). Each sample was measured 5 times and the results presented are the arithmetic means of n=4. The TEM analyses were performed in a Jeol JEM 1400 transmission electron microscope (Tokyo). The samples were mounted on 300 mesh formvar copper grids, stained with uranyl acetate 1%. Images were digitally recorded using a Gatan SC 1000 ORIUS CCD camera (Warrendale, PA, USA), and photomontages were performed using Adobe Photoshop CS software (Adobe Systems, San Jose, CA), at the HEMS/IBMC—Institute for Molecular and Cell Biology (IBMC) of the University of Porto, Portugal.

Results:

Our results indicated that the exosomes had an average size of 140 nm (FIG. 2A), a negative zeta potential (−30 mV) (FIG. 2B), and a spherical morphology (FIG. 2C.1 and 2C.2). In addition, these exosomes expressed the markers CD9, CD81 and HSC70 as analyzed by FACS (FIG. 2D).

There was no significant difference between the biophysical characteristics of exosomes secreted in hypoxia and that of those secreted in normoxia (control conditions). However, the data suggested that hypoxia stimulated increased secretion of exosomes by UCBMNCs, as observed from the increased protein content (FIG. 2E), which are normalized to the same number of secreting cells and re-suspended in equivalent final volumes.

### Example 1.2: Exosome Uptake Kinetics by Skin Cells

1.2.1 Percentage of Skin Cells

### Experimental Procedure

Skin cells uptake exosomes derived from UCBMNC, however the uptake kinetics and magnitude is dependent on the cell type. To demonstrate the differences in the exosome uptake kinetics by skin cells, exosomes were initially labeled with a fluorescent dye (NBD-DPPE and Syto green). Briefly, NBD-DPPE (16 μM in ethanol) or Syto Green (10 μM in DMSO) dyes were added to an exosome suspension (100 μL; protein concentration ranging from 60 to 70 μg/mL)(DMS0 and EtOH<1%) and incubated at 37° C. for 30 min. After blocking the reaction with bovine serum albumin (BSA, 1%, w/v) the exosomes were washed with PBS (6 mL) and the free dye was removed by ultracentrifugation at 100,000g for 1 h. The exosomes were then re-suspended in PBS (100 μL). This procedure was always performed with fresh exosomes. The labeled exosomes were used immediately in the internalization experiments.

We evaluated the percentage of skin cells able to uptake UCBMNC-derived exosomes after 4 h of incubation, by flow cytometry. HUVECs (80,000 cells/well), HaCaT cells (160,000 cells/well) and fibroblasts (50,000 cells/well) were seeded in a 24 well-plate and left to grow for 18-24 h. The confluent cells were incubated with NBD-DPPE labeled exosomes for 4 h in cell culture media without fetal bovine serum (FBS), at final concentrations of 1, 2 and 3 μg/mL. Afterwards, cells were washed once with PBS, detached with trypsin (HUVEC and NDHF) or tripLEm Express (HaCaT), washed twice in PBS and analyzed by flow cytometry on a FACS-Calibur instrument integrated with Cell-Quest software (BD Biosciences).

Results:

Exosomes are not cytotoxic to endothelial cells, for concentrations up to 10 μg/mL (FIG. 3A).

Our results also showed that for concentrations of exosomes between 1 and 3 μg/mL, higher internalization levels were observed for higher concentrations of exosomes (FIG. 3B). In addition, both keratinocytes and fibroblasts were more permissive to exosome internalization than endothelial cells.

1.2.2: Effect of Incubation Time

### Experimental Procedure

Next, we evaluated the influence of incubation time and concentration of exosomes in the uptake kinetics of exosomes by skin cells using confocal microscopy (FIG. 3C). The snapshots (FIG. 3C) were acquired with a Zeiss LSM 710 confocal scanning equipment, using the excitation wavelengths of, 405 and 488 and a Plan-Apochromat 40×/1.4 oil immersion objective. Kinetic profile was also obtained by analyzing the cytoplasm intensity at different time points for 24 h, using high-content microscopy (In Cell Analyser, GE Healthcare). The microscope is equipped with heat incubator which was set at 37° C. and a CO2 supply set to 5%. For live cell microscopy experiments, HUVEC cells were grown for 24 h in an IBIDI™ μ-Slide angiogenesis plate. Syto® RNASelectm labeled exosomes (1-20 μg/ml) were added to confluent cells in exo-depleted medium and incubated for different time points. Cells were washed two times with PBS and kept in FBS containing medium during the course of the experiments.

Results:

Our results showed that in the three types of cells tested, exosomes were located in the overall cytoplasm. Accumulation of exosomes in the cell cytoplasm peaked at approximately 16 h after contact (FIG. 3D).

1.2.3: Functional Effects of Exosomes

### Experimental Procedure

To determine functional effects of exosomes on skin cells, we evaluated whether exosome internalization could induce (i) cell survival under ischemic conditions, (ii) cell proliferation, (iii) cell migration and (iv) endothelial tube formation (FIGS. 4 and 5). For cell survival experiments, HUVECs (1×10⁴) were cultured in EGM-2 (Lonza), with 2% FBS, NDHFs (5×10³) and HaCaT (2×10⁴) were cultured in DMEM (Invitrogen), with 0.5% Penstrep and 10% FBS, in 96 plates. After 20 h, cells were washed with PBS and incubated with exo-depleted medium alone (100 μL) or with exosomes (100 μL, containing 1 μg/mL, 2 μg/mL, 3 μg/mL or 5 μg/mL) for 48 h. Then cells were incubated in ischemia conditions for 48 h (HUVECs in EGM-2 without VEGF and FBS; and HaCaT in DMEM without FBS at 37° C., 5% CO2, 0.1% O2) or 72 h (fibroblasts in DMEM without FBS at 37° C., 5% CO2, 0.1% O2). At the end the cells were monitored by high-content microscopy and cell viability was quantified by hoescht/propidium iodide (PI) staining. Cell survival was calculated according to the following equation: (Cell survival_{treatment group}−Cell survival_{control group})/Cell survival_{control group}.

The following procedure was adopted to demonstrate the proliferation inductive properties of exosomes. HUVECs (2.5×10³) were cultured in EGM-2 (Lonza), with 2% FBS, fibroblasts (5×10³) and HaCaT (2.5×10³) were cultured in DMEM (Invitrogen), with 0.5% Penstrep and 10% FBS, in 96 plates. After 20 h, cells were stained with Hoechst (33342, Life Technologies) for 15 min, washed with PBS and incubated in exo-depleted medium alone (100 μL) or with exosomes (1 μg/mL, 2 μg/mL or 3 μg/mL) for 24 h. Then the medium was changed and cells were incubated (37° C., 5% CO2, 0.1% O2) in exo-depleted medium (200 μL) for 72 h. Cells were stained with Hoechst and photographed (IN Cell, GE Healthcare) every 24 h and cell number was calculated by counting cell nuclei. Proliferation ratio was obtained by dividing the cell number at 72 h after treatment by the number of cells at the time of the incubation with exosomes (Time zero—T0).

Fibroblasts (1×10⁴) and HaCaT cells (2×10⁴) were cultured in DMEM (Invitrogen), with 0.5% Pen Strep and 10% FBS, in 96 plates. After 20 h, a vertical scratch was made with a 200 μl pipette tip in the middle of the well and cells were washed with PBS. Then, exo-depleted medium alone (100 μL) or supplemented with exosomes (1 μg/mL, 2 μg/mL or 3 μg/mL) was added to the cells and images of the wells (time zero T0) were taken in In Cell Analyzer (GE Healthcare).

After 4 h, further exo-depleted medium was added (100 μL) and cells incubated for 18 h. Afterwards, cell media was changed to exo-depleted DMEM with 0.5% FBS, and cells were incubated at 37° C., 5% CO2, 20% O2 for 48 h. Pictures were taken 48 h after scratch and wound area measured in AxioVision software 4.6.3 (Carl Zeiss Imaging Solutions GmbH).

To determine the effect of exosomes on tube formation in endothelial cells, HUVECs (5×10⁴/well, passage 3-5) were cultured in EGM-2 (Lonza) in 24 well plate over-night. After 20 h, cells were incubated in 200 μL of exo-depleted medium alone and with exosomes (1 μg/mL, 2 μg/mL, 3 μg/mL and 5 μg/mL). After 48 h cells were trypsinized and seeded in EBM-2 on top of the polymerized Matrigel (BD Biosciences, 10 μL per well, 30 minutes at 37° C.) in a 15-well ibidi plate. Twelve hours after cell seeding, cells were photographed by phase contrast microscopy and tube formation was evaluated and quantified using WimTube software (Wimasis).

Results:

Our results showed that exosomes isolated from UCBMNCs cultured in hypoxic conditions (Exo_Hyp), but not in normoxic conditions (Exo_Nor), were very efficient in inducing endothelial cell survival cultured in ischemic conditions for 48 h (FIG. 4A-4D).

According to our results, the survival peaked for concentrations of exosomes of 2 μg/mL. Interestingly, although both Exo_Hyp and Exo_Nor induce keratinocyte survival cultured in ischemic conditions they had no effect in the survival of fibroblasts. Therefore, our results indicate that the pro-survival of exosomes is cell and concentration dependent.

Our results showed that exosomes induced the proliferation of fibroblasts at concentrations of 1 and 2 μg/mL for Exo_Hyp and 2 and 3 μg/mL for Exo_Nor (FIG. 4E-4H). Interestingly, exosomes had no significant effect in the proliferation of endothelial cells and keratinocytes, showing one more type that exosome-induced properties are cell and concentration dependent.

Our results showed that exosomes, particularly Exo_Hyp, significantly increased mobility of fibroblast and keratinocyte cells, indicating that these exosomes also exhibit pro-migratory bioactivity (FIG. 5A-5C).

Our results showed that UCBMNCs-Exo significantly increased the total number of vascular tubes and the complexity of the vascular network formed, as observed from the augmented number of branching points (FIG. 5D).

### Example 2. Characterization of the RNA Content of UCBMNCs-Exo

### Experimental Procedure

The RNA content of UCBMNCs-Exo was characterized by RNA deep-sequencing. UCBMNCs-Exo secreted in hypoxia and normoxia from two different donors were used. The following methodology was used.

Total RNA was extracted from the exosomes pellet with the miRCURY RNA Isolation Kit—Cell & Plant (EXIQON) using the Lysate Preparation from Cultured Animal Cells protocol. The amount and quality of total RNA and small RNA were assessed using the RNA 6000 Pico kit and the Small RNA kit in the Agilent 2100 Bioanalyzer (Agilent Technologies), respectively. Samples proceeded for cDNA library preparation using both Whole Transcriptome (WT) and Small RNA libraries protocols described in Ion Total RNA-Seq Kit v2 User Guide (Life Technologies).

All procedures were carried out according to manufacturer's instructions, except the RNA fragmentation step that was skipped in the preparation of the whole transcriptome libraries since the starting material was already of the required length.

Briefly, 2.1-4.5 ng of small RNA and 20 ng of total RNA were used to construct the small and WT libraries, respectively. RNA adaptors were ligated at 16° C. for 16 h to small and at 30° C. for 1 h to WT libraries. The first and second cDNA strands were synthesized, and purified cDNA was then amplified with specific barcoded primers by PCR and the resulting fragments selected for the correct size with magnetic beads. The cDNA libraries were quantified, and their quality assessed with the Agilent High Sensitivity DNA Kit for WT RNA libraries and the Agilent DNA 1000 kit for small libraries in the 2100 Bioanalyzer (Agilent Technologies). Two pools of barcoded libraries (WT and small RNA) were prepared and clonally amplified by emulsion PCR using the Ion PI Template OT2 200 kit v2 and the Ion OneTouch 2 System (Life Technologies), and the positive Ion Sphere Particles enriched in the Ion OneTouch ES machine (Life Technologies). Finally, the positive spheres were loaded into a single Ion PI chip v2 and sequenced in the Ion Proton System (Life Technologies) at Genoinseq (Cantanhede, Portugal). All procedures were carried out according to manufacturer's instructions. Ion Proton adapter sequences and low-quality bases were trimmed using the Torrent Suite software (Life Technologies).

Sequence reads were then filtered by length, where reads with less than 15 bp were removed from the small library and 18 bp for the large library. Next, rRNA, tRNA, miRNA, mRNA and ncRNA were identified by aligning the reads from each sample to their specific libraries (rRNA: 5S—NR_023379.1, 5.8S—NR_003285.2, 18S-NR_003286.2, 28S—NR_003287.2; tRNA: library of tRNA extracted from UCSC; mir: mirbase; mRNA: CDS library from Ensembl; ncRNA library from Ensembl and ENCODE) using TMAP version 4.1.

Next, we verified the expression of the 15 miRNAs by qRT-PCR in exosomes collected from 10 individuals (Figure. 6C). After isolation of total RNA of exosomes secreted in hypoxia and normoxia by mononuclear cells of 10 different donors, all the miRNAs in the sample were polyadenylated and a cDNA library was constructed using the NCode™ miRNA first-strand cDNA synthesis and qRT-PCR Kit (Invitrogen™, Life Technologies, Carlsbad, California, USA) and the expression of the 15 most expressed microRNAs, as determined by the RNASeq data, was confirmed by qRT-PCR using the Fluidigm dynamic array in the BioMark™ system (Fluidigm corporation, South San Francisco, CA, USA), following the manufacturer's instructions. Specific primers for each microRNA were designed accordingly to NCode kit instructions and U6 snRNA was used as endogenous control (Wang Y, et al, 2015).

Results:

Our results showed that both Exo-Hyp and Exo_Nor have a large pool of miRNAs (FIG. 6A). In terms of total reads, the 30 most representative miRNAs for both exosomes were: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

The accession number and mature sequences of the 30 most representative miRNAs are summarized in Table I below.

TABLE I
Mature microRNA in UCBMNCs exosomes, miRBASE accession
number and sequence

| # | Name | Accession number | Mature sequence |
|---|---|---|---|
| 1 | hsa-mir-150-5p | MIMAT0000451 | UCUCCCAACCCUUGUACCAGUG |
| 2 | hsa-mir-16-5p | MIMAT0000069 | UAGCAGCACGUAAAUAUUGGCG |
| 3 | hsa-mir-142-3p | MIMAT0000434 | UGUAGUGUUUCCUACUUUAUGGA |
| 4 | hsa-mir-223-3p | MIMAT0000280 | UGUCAGUUUGUCAAAUACCCCA |
| 5 | hsa-let-7g-5p | MIMAT0000414 | UGAGGUAGUAGUUUGUACAGUU |
| 6 | hsa-mir-21-5p | MIMAT0000076 | UAGCUUAUCAGACUGAUGUUGA |
| 7 | hsa-let-7f-5p | MIMAT0000067 | UGAGGUAGUAGAUUGUAUAGUU |
| 8 | hsa-mir-19b-3p | MIMAT0000074 | UGUGCAAAUCCAUGCAAAACUGA |
| 9 | hsa-let-7a-5p | MIMAT0000062 | UGAGGUAGUAGGUUGUAUAGUU |
| 10 | hsa-mir-26a-1-5p | MIMAT0000082 | UUCAAGUAAUCCAGGAUAGGCU |
| 11 | hsa-mir-20a-5p | MIMAT0000075 | UAAAGUGCUUAUAGUGCAGGUAG |
| 12 | hsa-miR-181a-5p | MIMAT0000256 | AACAUUCAACGCUGUCGGUGAGU |
| 13 | hsa-miR-451a | MIMAT0001631 | AAACCGUUACCAUUACUGAGUU |
| 14 | hsa-miR-23a-3p | MIMAT0000078 | AUCACAUUGCCAGGGAUUUCC |
| 15 | hsa-miR-342-3p | MIMAT0000753 | UCUCACACAGAAAUCGCACCCGU |
| 16 | hsa-miR-191-5p | MIMAT0000440 | CAACGGAAUCCCAAAAGCAGCUG |
| 17 | hsa-miR-103a-3p | MIMAT0000101 | AGCAGCAUUGUACAGGGCUAUGA |
| 18 | hsa-miR-15a-5p | MIMAT0000068 | UAGCAGCACAUAAUGGUUUGUG |
| 19 | hsa-miR-142-5p | MIMAT0000433 | CAUAAAGUAGAAAGCACUACU |
| 20 | hsa-miR-146a-5p | MIMAT0000449 | UGAGAACUGAAUUCCAUGGGUU |
| 21 | hsa-miR-19a-3p | MIMAT0000073 | UGUGCAAAUCUAUGCAAAACUGA |
| 22 | hsa-miR-15b-5p | MIMAT0000417 | UAGCAGCACAUCAUGGUUUACA |
| 23 | hsa-miR-26b-5p | MIMAT0000083 | UUCAAGUAAUUCAGGAUAGGU |
| 24 | hsa-miR-30d-5p | MIMAT0000245 | UGUAAACAUCCCCGACUGGAAG |
| 25 | hsa-miR-146b-5p | MIMAT0002809 | UGAGAACUGAAUUCCAUAGGCU |
| 26 | hsa-miR-106b-5p | MIMAT0000680 | UAAAGUGCUGACAGUGCAGAU |
| 27 | hsa-miR-29a-3p | MIMAT0000086 | UAGCACCAUCUGAAAUCGGUUA |
| 28 | hsa-miR-17-5p | MIMAT0000070 | CAAAGUGCUUACAGUGCAGGUAG |
| 29 | hsa-miR-29b-3p | MIMAT0000100 | UAGCACCAUUUGAAAUCAGUGUU |
| 30 | hsa-miR-101-3p | MIMAT0000099 | UACAGUACUGUGAUAACUGAA |

The sequences of the forward primers used for qRT-PCR for the mature miRNAs and RNU6 are summarized in Table II below.

TABLE II
Mature microRNA and RNU6 accession numbers (miRBase and
GenBank) and sequences of the forward primers used for qRT-PCR

| # | Name | Accession number | Forward Primer |
|---|---|---|---|
| 1 | hsa-mir-150-5p | MIMAT0000451 | TCTCCCAACCCTTGTACC |
| 2 | hsa-mir-16-5p | MIMAT0000069 | TAGCAGCACGTAAATATTG |
| 3 | hsa-mir-142-3p | MIMAT0000434 | TGTAGTGTTTCCTACTTTATGGA |
| 4 | hsa-mir-223-3p | MIMAT0000280 | TGTCAGTTTGTCAAATACCC |
| 5 | hsa-let-7g-5p | MIMAT0000414 | TGAGGTAGTAGTTTGTACAGTT |
| 6 | hsa-mir-21-5p | MIMAT0000076 | TAGCTTATCAGACTGATGTTGA |
| 7 | hsa-let-7f-5p | MIMAT0000067 | TGAGGTAGTAGATTGTATAGTT |
| 8 | hsa-mir-19b-3p | MIMAT0000074 | TGTGCAAATCCATGCAAAA |
| 9 | hsa-let-7a-5p | MIMAT0000062 | TGAGGTAGTAGGTTGTATAGTT |
| 10 | hsa-mir-26a-1-5p | MIMAT0000082 | TTCAAGTAATCCAGGATAGGCT |
| 11 | hsa-mir-20a-5p | MIMAT0000075 | TAAAGTGCTTATAGTGCAGGTAG |
| 12 | hsa-miR-181a-5p | MIMAT0000256 | AACATTCAACGCTGTCGGG |
| 13 | hsa-miR-451a | MIMAT0001631 | AAACCGTTACCATTACTGAGTT |
| 14 | hsa-miR-23a-3p | MIMAT0000078 | ATCACATTGCCAGGGATT |
| 15 | hsa-miR-342-3p | MIMAT0000753 | TCTCACACAGAAATCGCAC |
| Ctr | RNU6 | NR_004394 | ACACGCAAATTCGTGAAG |

Our analysis also confirmed hsa-miR-150-5p as the most expressed with, roughly, more than 4 fold the expression of the next most expressed microRNA, hsa-miR-342a-3p (FIG. 6C).

A previous report (Gray et al., 2015) has identified 7 miRNAs that were differentially expressed in exosomes collected from cardiac progenitor cells cultured in normoxia and hypoxia: miR-292, miR-210, miR-103, miR-17, miR-199a, miR-20a and miR-15b. It is important to note that most of the miRNAs identified in this patent (with the exception of miR-20a and miR-15b) are new and not previously disclosed.

### Example 3. UCBMNCs-Exo Accelerate Wound Healing In Vivo

### Experimental Procedure

In Type 1 model, diabetes mellitus was induced by intraperitoneal injection of streptozotocin (Sigma-Aldrich) in male C57BL/6 mice (10-12 week-old), purchased from Charles River (Wilmington, MA, USA).

For a Type 2 diabetes model, db/db genetically modified mice were acquired from Charles River (Wilmington, MA, USA).

After confirming that glucose levels were greater than 300 mg/dL (six to eight weeks following diabetes induction in the Type 1 model), the animals were anesthetized, the dorsolumbar skin shaved and disinfected, and two 6 mm-diameter dorsal full-thickness excisional wounds were created with a sterile biopsy punch in each animal.

The wounds were then covered with PBS (10 μL; two times per day), Exo_Hyp (10 μL of an exosome suspension at 2 μg/mL; two times per day; therefore, 0.4 μg per wound for the a 10 days overall treatment) or PDGF-BB (100 μg/mL, Peprotech; 4 μg/cm²; once a day).

After surgery, the animals were maintained in individual cages with food and water ad libitum and in a temperature and humidity-controlled environment. The wounds were measured everyday over 10-15 days. At the end of the study, animals were sacrificed by cervical dislocation and 10 mm-diameter skin biopsies were collected for further analysis.

Results:

Our results showed that exosome treatment significantly accelerated the rate of healing of Type 1 diabetic excisional wounds (FIGS. 7A and 7B). Acceleration of wound healing (smaller wound size) was observed from as early as day 2, which was statistically significant in Donor 2. After 10 days, a consistent 55% improvement of wound closure upon application of UCBMNCs-Exo was observed (size of the treated wound is half the size of the control), independently of the donor. The same treatment regimen was applied to normal mice, and acceleration of wound closure, significant from day 3, leading to a general improvement of 39% at day 10 was observed (FIG. 7C).

In the Type 2 diabetes mouse model (db/db genetic model), results show UCBMNCs-Exo also accelerated wound healing, exhibiting a significant effect at day 2, a 20% improvement at day 10, and a total time of healing of 17 days which is 4 days sooner than the control treatment (FIG. 7D).

These animals were also treated with the active agent of a known product commercialized for the treatment of human diabetic ulcers (PDGF-BB), at the recommended regimen (once per day, maximum concentration recommended). Surprisingly, UCBMNCs-Exo were significantly more efficient, as they start to accelerate wound closure earlier (day 2, compared to day 5), exhibited higher improvement at day 10 (20% compared to 10% with PDGF-BB), and totally closed the wounds sooner (day 17 compared to day 18 with PDGF-BB) (FIG. 7D). Interestingly, when UCBMNC-Exo are applied twice daily in one wound of diabetic mice, wound healing is accelerated in the control distant wound during the first days, suggesting that they also stimulate healing in distant sites (FIG. 7F).

Histologic analysis of the wounds indicates that UCBMNCs-Exo treatment increased re-epithelization, stimulated formation of fibrotic tissue, and was associated with increased cell infiltrations, which may be indicative of augmented infiltration of macrophages, essential for wound healing (FIG. 7E).

### Example 4. MiR-150-5p Enhances Fibroblasts Survival to Ischemia and Keratinocyte Migration In Vitro

### Experimental Procedure

For the in vitro assays, endothelial cells, fibroblasts and keratinocytes were transfected with miR-150-5p (5 nM, miRIDIAN microRNA Human hsa-miR-150-5p mimic MIMAT0000082, Dharmacon, Lafayette, Colorado, EUA) and Lipofectamine® RNAiMAX transfection reagent (Invitrogen™) in 100 μl of complete medium for 48 h (Cell Survival and Tube formation assay) or 24 h (cell proliferation and scratch assay).

Transfection with only Lipofectamine was used as a negative control.

Vascular endothelial growth factor (VEGF, 50 ng/mL) and platelet derived growth factor (PDGF-BB, 50 ng/mL) were used as positive controls.

All the in vitro assays were carried out as described for exosomes bioactivity assessment.

Results:

Our results show that fibroblasts transfected with miR-150-5p have higher survival after 72 h of ischemia than non-transfected cells (FIG. 8A). The same was not true for endothelial cells and keratinocytes. We extended our analyses to migration, proliferation and angiogenesis assays. Keratinocytes transfected with miR-150-5p, but not fibroblasts, had improved migration properties, as assessed by a scratch assay. No statistical differences were observed for cells regarding proliferation and angiogenesis.

### Example 5. MiR-150-5p Enhances Wound Healing In Vitro

We evaluated the in vivo effect of hsa-miR-150-5p in a non-disease wound-healing model.

### Experimental Procedure

Six Male C57BL/6 wild-type mice (8 week-old), purchased from Charles River (France) and weighing between 20 and 30 g, were anesthetized and two full-thickness excision of 6 mm diameter each and 5 cm apart were performed with a sterile biopsy punch in the dorsum of each animal.

A single dose of miR-150-5p (5 μM), complexed with DharmaFECT 1 (120 μL) siRNA transfection reagent (Dharmacon, Lafayette, Colorado, EUA), was subcutaneously injected to the wound edge (2×40 μL) immediately after the wound creation.

A scramble miR (microRNA Mimic Negative Control, Dharmacon, Lafayette, Colorado, EUA) and PBS were used as negative controls.

Animals were kept in individuals cages with food and water ad libitum, and wound area was observed daily during the total period of the experiment. The wound area was traced every day onto acetate paper to follow the rate of wound closure up to 10 days post wounding. The wound size was determined with the ImageJ software (NIH). The data was presented as percentage of original wound (day 0). The animals were sacrificed 10 days post-wounding.

Results:

Our results showed that wounds treated with hsa-miR-150-5p healed faster than wounds treated with negative miR-control (scramble) and non-treated (PBS) wounds (FIG. 8E).

It is interesting to note that hsa-miR-150-5p is a mononuclear specific miR (Li, et al, 2013; Allantaz, et al, 2012; Zhang, et al, 2010). Previous studies have shown that hsa-miR-150-5p targets VEGF and has both anti-angiogenic and pro-angiogenic activities (He, et al, 2016; Yang, et al, 2015; Li, et al, 2013). Furthermore, hsa-miR-150-5p has been also described as being related with endothelial cell migration (Yang, et al, 2015; Zhang, et al, 2010) and cell proliferation and apoptosis (Gu, et al, 2014; Shi, et al, 2007). Our results show for the first time the involvement of hsa-miR-150-5p as a wound healing inductive miRNA.

### REFERENCES

Allantaz F., et al. (2012). Expression profiling of human immune cell subsets identifies miRNA-mRNA regulatory relationships correlated with cell type specific expression. PLoS One 7(1):e29979.
Abbade L. P. and Lastoria S. (2005). Venous ulcer: epidemiology, physiopathology, diagnosis and treatment. International Journal of Dermatology, 44:449-456. doi: 10.1111/j.1365-4632.2004.02456.x.
Bian S. et al. (2014). Extracellular vesicles derived from human bone marrow mesenchymal stem cells promote angiogenesis in a rat myocardial infarction mode. Journal of Molecular Medicine, 92:387-397. doi:10.1007/s00109-012-1110-5.
Boulton A. J. et al. (2005). The global burden of diabetic foot disease. Lancet, 366(9498):1719-24.
Crapnell, K. et al. (2013). Growth, differentiation capacity, and function of mesenchymal stem cells expanded in serum-free medium developed via combinatorial screening. Experimental Cell Research, 319:1409-1418.
Eberhardt R. T. and Raffetto J. D. (2005). Chronic venous insufficiency. Circulation, 10; 111(18):2398-409.
Erices, A., Conget, P., and Minguell, J. J. (2000). Mesenchymal progenitor cells in human umbilical cord blood. British Journal of Haematology, 109:235-242.
Gang, E. J. et al. (2004). Skeletal myogenic differentiation of mesenchymal stem cells isolated from human umbilical cord blood. Stem Cells, 22:617-624.
Gottrup F. (2004). A specialized wound-healing center concept: importance of a multidisciplinary department structure and surgical treatment facilities in the treatment of chronic wounds. American Journal of Surgery, 187(5A):38S-43S.
Goutos I. et al. (2015). Diabetes mellitus and burns. Part I-basic science and implications for management. International Journal of Burns and Trauma, 5(1):1-12.
Gray et al. (2015). Identification of Therapeutic Covariant MicroRNA Clusters in Hypoxia-Treated Cardiac Progenitor Cell Exosomes Using Systems Biology. Circulation Research, 116: 255-263. doi: 10.1161/CIRCRESAHA.116.304360.
Guo S. and DiPietro L. A. (2010). Factors Affecting Wound Healing. Journal of Dental Research, 89(3):219-29. doi: 10.1177/0022034509359125.
Gu, X. et al. (2014). Down-regulation of miR-150 induces cell proliferation inhibition and apoptosis in non-small-cell lung cancer by targeting BAK1 in vitro. Tumor Biol, Jun; 35(6):5287-93
He, Q-W. et al. (2016). MiR-150 regulates poststroke cerebral angiogenesis via vascular endothelial growth factor in rats. CNS Neurosci & Therap. 22: 507-517.
Lai, R. C. et al. (2010). Exosome secreted by MSC reduces myocardial ischemia/reperfusion injury. Stem Cell Research, 4:214-22.
Lai, R. C., Chen, T. S., and Lim, S. K. (2011). Mesenchymal stem cell exosome: a novel stem cell-based therapy for cardiovascular disease. Regenerative Medicine, 6:481-492.
Li, J. et al. (2013). Microvesicle-mediated transfer of microRNA-150 from monocytes to endothelial cells promotes angiogenesis. J Biol Chem. August 9; 288(32):23586-96.
Mathivanan, S. and Simpson, R. J. (2009). ExoCarta: A compendium of exosomal proteins and RNA. Proteomics, 9:4997-5000.
O'Loughlin, A. J., Woffindale, C. A., and Wood, M. J. A. (2012). Exosomes and the Emerging Field of Exosome-Based Gene Therapy. Current Gene Therapy, 12:262-274.
Nunan R., Harding K. G., and Martin P. (2014). Clinical challenges of chronic wounds: searching for an optimal animal model to recapitulate their complexity. Disease Models and Mechanisms, 7(11):1205-13. doi: 10.1242/dmm.016782.
Pittenger, M. F. et al. (1999). Multilineage potential of adult human mesenchymal stem cells. Science, 284:143-147.
Raposo, G. and Stoorvogel, W. (2013). Extracellular vesicles: Exosomes, microvesicles, and friends. Journal of Cell Biology, 200:373-383.
Reddy M., Gill S. S., and Rochon P. A. (2006). Preventing pressure ulcers: a systematic review. JAMA, 296(8):974-84.
Sahoo, S. et al. (2011). Exosomes From Human CD34+Stem Cells Mediate Their Proangiogenic Paracrine Activity. Circulation Research, 109:724-728.
Sen C. K., et al. (2009). Human skin wounds: a major and snowballing threat to public health and the economy. Wound Repair and Regeneration: Official Publication of the Wound Healing Society and the European Tissue Repair Society, 17(6):763-71. doi: 10.1111/j.1524-475X.2009.00543.x.
Shabbir et al. (2015). Mesenchymal stem cell exosomes induce the proliferation and migration of normal and chronic wound fibroblasts, and enhance angiogenesis in vitro. Stem Cells and Development, 24(14): 1635-1647. doi: 10.1089/scd.2014.0316.
Shi, X., et al. (2007). An androgenregulated miRNA suppresses Bak1 expression and induces androgen-independent growth of prostate cancer cells. Proc Natl Acad Sci USA. 104(50), 19983-8.
Thery, C. et al. (2006). Isolation and characterization of exosomes from cell culture supernatants and biological fluids. Curr Protoc Cell Biol., Chapter 3, Unit 3.22.
Velnar, T., Bailey, T., and Smrkoli, V. (2009). The Wound Healing Process: an Overview of the Cellular and Molecular Mechanisms. Journal of International Medical Research, 37:1528-1542.
Wang Y, et al, (2015). Exosomes/microvesicles from induced pluripotent stem cells deliver cardioprotective miRNAs and prevent cardiomyocyte apoptosis in the ischemic myocardium. Int J Cardiology, August 1; 192:61-9.
Yang, J. et al. (2015). Specific miRNA and its target in neutrophils after traumatic injury. Acta Biochim Biophys Sin, 2015, 47(9), 749-754.
Zhang, Y. et al. (2010). Secreted monocytic miR-150 enhances targeted endothelial cell migration. Mol Cell. July 9; 39(1):133-44.
Zhang B. et al. (2015). HucMSC-Exosome Mediated-Wnt4 Signaling Is Required for Cutaneous Wound Healing. Stem Cells, 33:2158-2168.
Zhang J. et al. (2015). Exosomes released from human induced pluripotent stem cells-derived MSCs facilitate cutaneous wound healing by promoting collagen synthesis and angiogenesis. Journal of Translational Medicine, 13:1-14. doi: 10.1186/s12967-015-0417-0.
Zhou, Y. et al. (2013). Exosomes released by human umbilical cord mesenchymal stem cells protect against cisplatin-induced renal oxidative stress and apoptosis in vivo and in vitro. Stem Cell Res Ther., 4:34.

1. A composition comprising exosomes secreted by umbilical cord blood mononuclear cells (UCBMNCs), wherein the UCBMNCs consist of lymphocytes and monocytes, and the secreted exosomes comprise one or more miRNA selected from a group of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

2. The composition comprising exosomes secreted by umbilical cord blood mononuclear cells (UCBMNCs) according to claim 1, wherein the exosomes comprise one or more miRNA selected from a group of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, and hsa-miR-146b-5p.

3. The composition comprising exosomes secreted by umbilical cord blood mononuclear cells (UCBMNCs) according to claim 2, wherein the exosomes comprise one or more miRNA selected from a group of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, and hsa-miR-146a-5p.

4. The composition comprising exosomes secreted by umbilical cord blood mononuclear cells (UCBMNCs) according to claim 3, wherein the exosomes comprise one or more miRNA selected from a group of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, and hsa-miR-342-3p.

5. The composition comprising exosomes secreted by umbilical cord blood mononuclear cells (UCBMNCs) according to claim 4, wherein the exosomes comprise one or more miRNA selected from a group of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, and hsa-miR-26a-1-5p.

6. The composition comprising exosomes secreted by umbilical cord blood mononuclear cells (UCBMNCs) according to claim 5, wherein the exosomes comprise one or more miRNA selected from a group of: hsa-miR-150-5p, hsa-miR-16-5p, hsa-miR-142-3p, hsa-miR-223-3p, hsa-let-7g-5p, and hsa-miR-21-5p,

7. The composition comprising exosomes secreted by umbilical cord blood mononuclear cells (UCBMNCs) according to claim 6, wherein the exosomes contain the miRNA hsa-miR-150-5p.

8. The composition according to claim 1, wherein the exosomes are enriched by the addition of one or more miRNA selected from a group of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p, thereby increasing the concentration of the miRNA in the exosomes.

9. The composition according to claim 8, wherein the exosomes are enriched by the addition of one or more miRNA selected from a group of: hsa-miR-181a-5p, hsa-miR-451a, hsa-miR-103a-3p, hsa-miR-15a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p, thereby increasing the concentration of the miRNA in the exosomes.

10. The composition according to claim 9, wherein the exosomes are enriched by the addition of one or more miRNA selected from a group consisting of: miRNA hsa-miR-150-5p, thereby increasing the concentration of the miRNA in the exosomes.

11. The composition according to claim 1, wherein the exosomes secreted by UCBMNCs express the markers CD9, CD34, CD81, and/or Hsc70.

12. The composition according to claim 1 wherein the concentration of the exosomes in the composition is of 0.01 μg/mL to 10 μg/mL.

13. The composition according to claim 12 wherein the concentration of the exosomes in the composition is of 1 μg/mL to 3 μg/mL.

14. The composition according to claim 13, wherein the concentration of the exosomes in the composition is of 0.5 μg/mL-1.5 μg/mL.

15. The composition according to claim 14, wherein the concentration of the exosomes in the composition is of 1 μg/mL, 0.01 μg/mL, 0.25 μg/mL, 0.5 μg/mL, 0.75 μg/mL, 1.0 μg/mL, 1.25 μg/mL, 1.5 μg/mL, 1.75 μg/mL, 2.0 μg/mL, 2.25 μg/mL, 2.5 μg/mL, 2.75 μg/mL, 3.0 μg/mL, 5.0 μg/mL, or 10 μg/mL.

16. The composition according to claim 1, wherein said composition is in the form of a liquid, a cream or a gel comprised in a pharmaceutical preparation or a dressing.

17. The composition according to claim 16, wherein the pharmaceutical preparation or the dressing is a graft material, a patch, a pad, a plaster, a film, a tape, an adhesive, a gel, a foam, a cream, a salve, a balm, a embrocation, an ointment, a poultice, an unction, an emollient, a liniment, a potion, an unguent, a lotion, a spray, a suspension, a powder, a syringe, a nebulizer or an aerosol container.

18. A method for promoting wound healing in a subject comprising contacting a wound of the subject with a composition of exosomes secreted by umbilical cord blood mononuclear cells (UCBMNCs), wherein the UCBMNCs consist of lymphocytes and monocytes, and the exosomes comprise one or more miRNA selected from a group of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p.

19. The method for promoting wound healing in a subject according to claim 18, wherein the wound is a skin open wound comprising an incision, a laceration, a tear, an abrasion, an avulsion or a surgical wound.

20. The method for promoting wound healing in a subject according to claim 18, wherein the wound is a burn wound comprising a thermal burn, a chemical burn or a radiation burn wound.

21. The method for promoting wound healing in a subject according to claim 18, wherein the wound is a skin wound or skin disease comprising acne, psoriasis, rosacea, dermatitis, eczema, impetigo, intertrigo, or folliculitis.

22. The method for promoting wound healing of a subject according to claim 18, wherein the wound is arterial ulcers, venous ulcers, diabetic ulcers and pressure ulcers, post-surgical ulcer, traumatic ulcer, mouth ulcer, diabetic foot ulcer or corneal ulcer.

23. A process for preparing a composition comprising exosomes secreted by umbilical cord blood mononuclear cells (UCBMNCs), wherein the UCBMNCs consist of lymphocytes and monocytes, and at least one of the secreted exosomes comprise one or more miRNA selected from a group consisting of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p, said process comprises the following steps:
a) Isolating the lymphocytes and monocytes from a total fraction of UCBMNCs by density gradient centrifugation using a solution comprising sodium diatrizoate (9.1% w/v) and polysaccharide (5.7% w/v) having a density of 1.077 g/ml, and
b) Isolating the exosomes secreted by umbilical cord blood mononuclear cells (UCBMNCs) by sequential centrifugation of the supernatant obtained in the previous step and resuspending the obtained pellet in a phosphate buffered saline solution (PBS).

24. The process for preparing a composition comprising exosomes secreted by umbilical cord blood mononuclear cells (UCBMNCs), according to claim 23, further comprising a step of adding to the isolated exosomes one or more miRNA selected from a group consisting of hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p, thereby increasing the concentration of the miRNA in the exosomes.

25. The process for preparing a composition comprising exosomes secreted by umbilical cord blood mononuclear cells (UCBMNCs), according to claim 23, wherein the isolated exosomes are exposed to ischemic conditions and/or hypoxic conditions previous to the isolation of step a).
